(19) United States
(12) Patent Application Publication
(10) Pub. No.: US 2003/0175745 A1
(43) Pub. Date: Sep. 18, 2003

(54) Peptides with growth inhibitory action

(76) Inventors: Cheryl H. Dean, Raleigh, NC (US); Mohammad A. Heidaran, Cary, NC (US); Catherine A. Spargo, Apex, NC (US); Perry D. Haaland, Chapel Hill, NC (US)

Correspondence Address: BECTON, DICKINSON AND COMPANY, 1 BECTON DRIVE, FRANKLIN LAKES, NJ, 07417-1880, US

(21) Appl. No.: 10/259,816

(22) Filed: Sep. 30, 2002

Related U.S. Application Data
(60) Provisional application No. 60/333,476, filed on Nov. 28, 2001.

(51) Int. Cl. C12Q 1/68; A61K 38/08; A61K 31/7072; A61K 31/337; A61K 31/704; A61K 31/4745

(52) U.S. Cl. 435/6; 514/17; 514/34; 530/329; 514/449; 514/49; 514/161; 514/283

(57) ABSTRACT

Peptides and peptide compositions are identified which inhibit the growth of abnormal cells. In one embodiment, the peptides are useful for inhibiting the growth of cells dependent on autocrine activation of the PDGF receptor. Such peptides may be used in the treatment of cell proliferative disorders including cancer, fibrotic disorders, myeloproliferative diseases and blood vessel proliferative (angiogenic) disorders characterized by inappropriate PDGF receptor activity. A biomedical device is further disclosed which has associated with it a peptide or peptide composition according to the present invention.

### BACKGROUND OF THE INVENTION

1. Field of the Invention

This invention, in the field of biochemistry, cell biology and medicine, is directed to peptides and peptide compositions that inhibit the growth of abnormal cells, such as cells that grow due to autocrine activation of the PDGF receptor (PDGF-R). Such peptides are used in the treatment of cell proliferative disorders including cancer, fibrotic disorders, myeloproliferative diseases and blood vessel proliferative (angiogenic) disorders. The invention includes a biomedical device that has associated therewith is such an inhibitory peptide.

2. Description of the Background Art

Platelet-derived growth factor (PDGF) is a major protein mitogen for cells of mesenchymal origin, including fibroblasts, smooth muscle cells and glial cells. The protein is normally a 32 kDa heterodimer composed of an α and a β chain linked by disulfide bonds. In addition to the PDGF αβ heterodimer, two homodimeric forms of PDGF, referred to as αα and ββ and, have been identified that are composed of two α chains or two β chains. For reviews of PDGF, its structure, activity, receptors, etc., see, for example: Westermark, B et al., eds., Biology of Platelet-Derived Growth Factor., Basel, Karger, 1993; Platelet-Derived Growth Factor, at the web address, rndsystems.com/asp/g_sitebuilder.asp?bodyId=220.

The first event to occur in PDGF-mediated mitogenesis is the binding of PDGF to its cell surface receptor (PDGF-R) (Bonner, J. C. (1994) Ann. N.Y. Acad. Sci. 737:324; Claesson-Welsh, L. (1994) J. Biol. Chem. 269:32023; Hart, C E et al., (1990) J. Invest. Dermatol. 94:53S). This binding triggers a variety of cellular responses which include activation of the receptor tyrosine kinase, increased phosphatidylinositol turnover, activation of phospholipase A2, the enhanced expression of a particular group of genes, changes in cell shape, an increase in intracellular calcium concentration, changes in intracellular pH, as well as internalization and degradation of the receptor-bound PDGF. These changes arc followed by an increase in the rate of proliferation of cells displaying the PDGF-R.

PDGF has been implicated in arteriosclerosis, myeloproliferative disease, as well as in stimulating genes associated with oncogenic transformation of cells, including c-myc and c-fos. Therefore, PDGF antagonists would potentially be useful in controlling induction of cancer and the proliferation of tumor cells.

Due to the fact that the interaction of PDGF with cells is mediated, in part, by a specific receptor, PDGF-R, the PDGF-R is also an important component in mitogenic stimulation by PDGF. For this reason, an antagonist at the PDGF-R would be expected to control tumor induction or proliferation.

Several approaches have recently been taken to develop antagonists of PDGF or PDGF-R, or receptor interactions with other proteins as described in further detail below.

Antibodies against PDGF have proven useful for inhibiting both the autocrine stimulation in simian sarcoma virus (SSV)-transformed cells (Johnsson A et al., Nature (1985) 317:438-440) and the arteriosclerotic process that occurs after de-endothelialization of the carotid arteries of rats Ferns G A et al., Science (1991) 253:1129-32. Moreover, a soluble form of the PDGF-R has been shown to bind and inactivate PDGF ((Tiesman, J. et al. (1993) J. Biol. Chem. 268:9621); Duan D S et al. (1991) J Biol Chem 266:413-4188) and could therefore potentially be used to inhibit PDGF action in vivo.

Furthermore, low molecular weight compounds that are competitive antagonists for PDGF binding to PDGF-R have been described, e.g., suramin, which inhibits PDGF binding to PDGF R at concentrations ranging from nM to μM (and is 100% inhibitory in the μM range). However, suramin is not specific enough to be clinically useful as a PDGF antagonist. Moreover, another low molecular weight compound, neomycin, at high concentrations inhibited the binding of PDGF-ββ to the α-type PDGF-R, but was not able to inhibit binding to the PDGF β receptor. However, this compound, which represents an antagonist of the α receptor type, has low potency, making it unsuitable for use in vivo.

Another approach has been to identify peptides affecting PDGF-R activities and receptor interactions with other proteins. To this end, U.S. Pat. No. 6,043,211 (L. T. Williams et al.) describes synthetic human PDGF-R peptides of 20 or fewer amino acid residues that are described as useful in medical diagnosis and drug therapies by affecting such PDGF-R activities and interactions. A disadvantage of using such long peptides is their high susceptibility to degradation at high temperatures and to the proteolytic action of serum proteases or cellular proteases. Therefore, polypeptides disclosed in the above patent would not be suitable for use with biomedical implants that are to be implanted for prolonged intervals (or permanently).

Thus, a need exists in the art to identify new peptides which affect the interaction between PDGF and PDGF-R and/or which affect PDGF-R interactions with other proteins. Specifically, it would be desirable to identify and use relatively short peptides that inhibit autocrine activation of PDGF-R as therapeutic agents for cell proliferative disorders, including cancers, which are characterized by inappropriate or undesirable PDGF-R activity. Furthermore, it would beneficial to provide a means for delivering such peptides to a selected site in vivo in the treatment of these disorders.

### SUMMARY OF THE INVENTION

The present invention provides an isolated peptide or polypeptide of no more than about 50 amino acid residues which, when contacted with cells in which a PDGF-R is activated in an autocrine manner, inhibits the growth of the cells, wherein the peptide or polypeptide comprises one or more amino acid sequences selected from the group consisting of KKKK (SEQ ID NO: 1), DDEEK (SEQ ID NO: 2), KLMSY (SEQ ID NO: 3), FFFKK (SEQ ID NO: 4), FFHPV (SEQ ID NO: 5), or (i) a combination thereof, (ii) a biologically active variant thereof having the same amino acid composition in a different sequence, (iii) or a biologically active substitution or addition variant. The above peptide or polypeptide preferably has no more than about 20 amino acids, preferably no more than about 10 amino acids. A preferred peptide is one selected from the group of peptides consisting of KKKK (SEQ ID NO: 1), DDEEK (SEQ ID NO: 2), KLMSY (SEQ ID NO: 3), FFFKK (SEQ ID NO: 4), and FFHPV (SEQ ID NO: 5).

A preferred polypeptide or peptide does not exceed about 50 amino acid residues. In other embodiments, the polypeptide or peptide has between about 45-50 residues, 40-45 residues, 35-40 residues, 30-35 residues, 25-30 residues, 20-25 residues, 15-20 residues, 10-15 residues or 5-10 residues.

Also included herein is a pentapeptide that falls within a parameter space defined by at least two physicochemical parameters of peptides SEQ ID NO:2-SEQ ID NO:5, that has the following properties: inhibits the growth of cells that expressing a PDGF-R that is activated for growth in an autocrine manner; has total charge of between −4 and +2; and has an MlogP value between about −8.5 and −2. More preferably the pentapeptide has a total charge between −4 and −2, and a MlogP value between about −7 and −3.5.

Also provided is a chemically synthesized peptide multimer comprising the above peptide 1, which multimer is disclosed in the Detailed Description sections below.

Another embodiment is a recombinantly produced peptide multimer comprising the above peptide or variant of, which multimer has the formula (P¹-Gly_{z})ₙ-P², which multimer is disclosed in the Detailed Description sections below.

The present invention provides an isolated nucleic acid molecule encoding (a) the polypeptide or peptide described above or any permuted sequence of SEQ ID NO:2-SEQ ID NO:5, or (b) the peptide multimer. The nucleic acid molecule may comprise one or more of SEQ ID NO:6-SEQ ID NO:341, inclusive.

Also included is an expression vector comprising the above nucleic acid molecule of operatively linked to a promoter, and, optionally, additional regulatory sequences that regulate expression of the nucleic acid in a eukaryotic cell, which vector is capable of expressing the peptide in a host cell. Preferred expression vectors are plasmids and viral vectors.

Peptides and nucleic acids of the present invention desirably inhibit the activity of a PDGF-R including receptor interactions with proteins other than PDGF. These peptides are useful for inhibiting autocrine stimulation of cells by PDGF that is mediated, at least in part, by binding to the PDGF-R. Preferably, the peptides also inhibit the activity of other members of the PDGF-R superfamily (see, for example, Qiu, F H et al., EMBO J. 1988, 7:1003-1011) such as PDGF-R and the PDGF-R-related kinase Flt, and KDR. An expression vector encoding a peptide as above and capable of being expressed in a host cell is also provided.

The present invention is also directed to a solid phase article comprising the polypeptide or peptide, or the multimer, described above, in contact with, preferably chemically linked to, a solid surface. The solid surface may comprise a synthetic polymer, natural polymer, or a combination thereof.

The article may further comprise an additional layer of a CAR material between the polypeptide or peptide and the surface. The CAR material is preferably (a) polyethylene glycol, (b) glyme, (c) a glyme derivative, (d) poly-HEMA, (e) polyisopropylacrylamide, (f) hyaluronic acid, (g) alginic acid or (h) a combination of any of (a)-(g).

The solid surface of the article preferably comprises a synthetic polymer selected from the group consisting of poly(hydroxyethyl methacrylate), poly(ethylene terephthalate), poly(tetrafluoroethylene), fluorinated ethylene, poly(dimethyl siloxane), and a combination thereof. When the solid surface comprises a natural polymer, it is preferably collagen, fibronectin, elastin, cellulose acetate, cellulose nitrate, polysaccharides, fibrin, gelatin, or combination thereof.

In the above article, the peptide may be chemically linked to the surface through a linker molecule.

This invention includes a biomedical device for inhibition of abnormal or undesired cell attachment, cell growth or both attachment and growth, comprising a biocompatible surface having chemically and/or physically associated with the surface a proliferation inhibiting amount of the peptide, polypeptide or combination above, the peptide multimer above, or a nucleic acid molecule encoding the peptide or polypeptide or multimer.

The above device may further comprise an additional layer of a CAR material between the polypeptide or peptide and the surface. The peptide or polypeptide may be impregnated in or coated on the surface. The peptide or polypeptide may be present as a controlled release composition.

In yet another embodiment is presented a therapeutic composition that inhibits the undesired growth of cells mediated by abnormal activation or activity of PDGF-R, comprising the above growth inhibitory peptide, polypeptide combination, peptide multimer or nucleic acid (expression vectors) and a therapeutically acceptable carrier or excipient. The abnormal activation may comprise autocrine activation of the PDGF-R.

Unwanted cell proliferation can result from inappropriate PDGF-R activity in any of a number of cell types including cancer cells, stromal cells surrounding cancer cells, endothelial cells and smooth muscle cells. The methods and compositions of the present invention are designed to inhibit unwanted cell proliferation of any cell type by altering the activity of the PDGF-R and/or its interactions with other proteins.

Also provided is a method of inhibiting cell proliferation comprising contacting cells undergoing undesired proliferation with an effective amount of the peptide, polypeptide, combination, multimer, or expression vector described above. The cell being inhibited may be a tumor or cancer cell, such as a carcinoma cell, an osteocarcinoma cell, a sarcoma cell, an osteosarcoma cell, a glioma cell, a melanoma cell, a myxoma cell, an adenoma cell, a neuroblastoma cell, or a rhabdomyoma-derived cell. The cell being inhibited may be a lung cell, a breast cell, a colon cell, a prostate cell, a kidney cell, an ovary cell, a testicular cell, a skin cell, a pancreatic cell, a thyroid cell, an adrenal cell, a pituitary cell, a brain cell, a muscle cell or a bone cell.

In the above methods of treatment, the contacting is preferably in vivo in a subject, but also may be in vitro.

The above therapeutic method may further comprise administering to the subject of a therapeutically effective amount of one or more agents or drugs selected from the group consisting of cisplatin, cyclophosphamide, VP-16, enoxaprin, angiopeptin, endostatin, paclitaxel, 5-fluorouracil, vinblastine, vincristine, an epothilone, angiostatin, hirudin, acetylsalicylic acid, and a thymidine kinase inhibitors.

A method of treating a subject suffering from a cell proliferative disorder, comprises contacting cells of the subject which are characterized by inappropriate PDGF receptor activity with an effective amount of a peptide, polypeptide, combination, or multimer as above or with a nucleic acid molecule encoding the peptide, polypeptide, or multimer, which nucleic acid is expressible in the cells.

In the above methods, the peptide, polypeptide or multimer may be in contact with, associated with or chemically linked to a biomedical implant. The biomedical implant comprises at least one of a natural polymer or a synthetic polymer.

Also included is a method of treating a subject who has a solid tumor, the cells of which are characterized by inappropriate PDGF receptor activity, the method comprising contacting tumor cells and/or cells surrounding tumor cells of the subject with an effective amount of a peptide, polypeptide or combination, with a peptide multimer, or with a nucleic acid molecule encoding the peptide or polypeptide which nucleic acid is expressible in the tumor or surrounding cells. The method may further comprising prior to the contacting step, the steps of surgically removing or debulking the solid tumor; and implanting a biomedical device that comprises the therapeutic material proximal to the site of the surgery.

### DESCRIPTION OF THE PREFERRED EMBODIMENTS

The present invention provides methods and compositions for treating a cell proliferation disorder characterized by inappropriate PDGF-R activity. Without being bound to any one theory, inhibition of unwanted cell proliferation may be brought about by altering the activity of the PDGF-R, such as inhibiting phosphorylation of the receptor, inhibiting the substrate or adapter protein binding to the receptor, or inhibiting downstream signaling events, thereby inhibiting PDGF-R activity.

Binding of PDGF to the PDGF-R induces receptor dimerization and allosteric changes that activate the intracellular Tyr kinase domains, resulting in receptor transphosphorylation and/or autophosphorylation on Tyr residues. Such phosphorylation stimulates a physical association of the activated receptor with target molecules, some of which are, in turn, phosphorylated allowing transmission of the signal to the cytoplasm. Other target molecules are not phosphorylated, but contribute to signal transduction by acting as docking or adapter molecules for secondary signal transducer proteins. The secondary signal transducer molecules generated by activated receptors result in a signal cascade that regulates cell functions including cell division (Fry, M. J. et al., Protein Science 2: 1785-1797, 1993).

“Cell proliferative disorder” or “cell proliferation disorder” refers to a disorder wherein unwanted cell proliferation of one or more types of cells in a multi-cellular organism occurs and results in harm (e.g., discomfort or disease or decreased life expectancy) to the organism. Cell proliferative disorders occur in animals including humans. These disorders can include any form of cancer, blood vessel proliferative (angiogenic) disorders, and fibrotic disorders. These disorders are not necessarily independent of one another. For example, a fibrotic disorder may be related to, or overlap with, a blood vessel disorder.

“Inappropriate PDGF-R activity” refers to one or more of the following: (1) abnormal PDGF-R expression wherein receptor is expressed in cells which normally do not express it; (2) abnormal PDGF expression by cells which normally do not express PDGF; (3) increased PDGF-R expression leading to unwanted cell proliferation; (4) increased PDGF expression leading to unwanted cellular proliferation; or (5) mutations leading to constitutive activation of a gene or gene encoding the PDGF-R which result in abnormal receptor expression. The determination of inappropriate or abnormal PDGF and PDGF-R expression, level or activity is determined by methods well known in the art.

Unwanted cell proliferation can result from inappropriate PDGF-R activity in various types of cells including cancer cells, cells surrounding a cancer cell (stromal cells), endothelial cells and smooth muscle cells. For example, increased PDGF-R activity in endothelial cells surrounding cancer cells may lead to an increased neovascularization of the tumor, thereby facilitating tumor growth and ultimately, metastasis. Therefore, inappropriate PDGF-R activity can contribute to a cell proliferative disorder in a number of ways including increasing the production of other growth factors (for example fibroblast growth factor, interleukin-1 alpha or vascular endothelial growth factor) causing abnormal cell growth and increased formation and spread of blood vessels in a solid tumor thereby enabling tumor growth and metastasis.

The present inventors have identified a set of inhibitory peptides that inhibit the growth of abnormal cells such as tumor cells. Useful peptides are those that include the following amino acid sequences: KKKK (SEQ ID NO: 1), DDEEK (SEQ ID NO: 2), KLMSY (SEQ ID NO: 3), FFFKK (SEQ ID NO: 4), FFHPV (SEQ ID NO: 5), and combinations thereof. The inhibitory action of the peptides provide a mechanistic basis for formulation of products, such as a biomedical device with growth inhibitory action. Such a device may be formulated by immobilizing such peptides in a two-dimensional or three-dimensional vehicle consisting of natural or synthetic polymers or a combination thereof.

In other aspects, the invention features novel compositions, such as therapeutic or pharmaceutical compositions that include one or more of the growth-inhibitory polypeptides, peptides, multimers or nucleic acids described herein.

Based on the identification of the 5 peptides as having desirable growth-inhibitory activity, the screening and determination of a parametric space defining additional peptides sharing the properties of one or of several of these peptides is carried out employing the methods and software described in Campbell, R et al., WO 01/07642, and Haaland et al., WO 02/02591, both of which are herein incorporated by reference. This permits definition of ranges of selected physicochemical parameters that define a parametric space within which additional peptides with desirable inhibitory properties would fall. This approach is described in more detail in the following sections.

As described in the foregoing documents, a relationship (e.g., mathematical) is determined between at least one parameter or descriptor (e.g., physical, chemical, biological and/or topological) of the five peptides (SEQ ID NO:1-SEQ ID NO:5 which were shown to have the measured indicia of a desired property, here cell growth inhibition. The relationship can be used as a predictor to identify additional peptides that are expected, based on their parameters, to give indicia of the measured property that satisfy a test requirement. Preferred parameters include molecular weight, charge, isoelectric point, total dipole moment, isotropic surface area, electronic charge index, and hydrophobicity of the whole peptide or individual amino acid. Any suitable topological parameter known in the art may be employed, such as those described by L. B. Kier and L. H. Hall, Molecular Connectivity in Structure-Activity Analysis, Research Studies Press, John Wiley & Sons, Letchworth England (1986); M. Johnson et al., Concepts and Applications of Molecular Similarity, John Wiley & Sons, New York (1990); and R. P. Sheridan et al., (1995) J. Chem. Inf. Comput. Sci, 35:310. The term “parameters” as used herein also encompasses the principle components of S. Hellberg et al., (1987) J. Med. Chem. 30:1126 (e.g., z₁, z₂, z₃).

Since growth inhibition is the selective test requirement here, the measured indicia of this property are compared for other peptides to be selected from a peptide library, for example Preferably indicia of growth inhibition that fall within a particular range of the five peptides described above are preferred

The relationship determined between the parameter(s) of the five peptides and the indicia of the growth inhibitory property can be determined by any method for describing the interaction between the activity and the structure of chemical compounds, for example, by quantitative structure-activity relationships (QSAR), nearest neighbor analysis, self-organizing maps, or other machine learning and statistical techniques.

In one embodiment, the relationship may be expressed in the form of ŷ₁=f(xᵢⱼ), where xᵢⱼ denotes a parameter, i ranges from 1 to n, where n represents the number of first peptides tested, j ranges from 1 to d, where d represents the number of parameters measured, and ŷ₁ represents an estimate of the measured first indicia of the property. The relationship represented by ŷ₁=f(xᵢⱼ) may be a parametric or non-parametric formula.

The relationship between the parameter(s) of the test compounds and the indicia of the measured property is based on a distance function between the parameters of the first tested compounds herein, the selected five peptides SEQ ID NO:1-SEQ ID NO:5 and the parameters of untested peptides, preferably, pentapeptides. The distance function can be expressed as d(x₁, x₂) between a first value of a parameter, x₁, of a first test compound and a second value of the same parameter, x₂, of a second untested compound. This relationship assigns to a second untested peptide an estimated indicia of the property that corresponds to the actually measured indicia determined for a first tested compound from the first test library if d(x₁, x₂)=d_{cutoff1}, where d_{cutoff1} is a cutoff distance for the first test compound. In other words, once a lead peptide, such as any of peptides SEQ ID NO:1-SEQ ID NO:5, is identified from the first test library, additional lead peptides can be determined based on an assumption that peptides that are close in parameter space will exhibit similar or better inhibitory activity. x₁ and x₂ may represent a single parameter or a set of parameters, i.e., x₁=x₁₁, x₁₂, x₁₃, x₁₄ . . . x₁ₖ and x₂=x₂₁, x₂₂, x₂₃, x₂₄ . . . x₂ₖ, where k≧1.

One specific example of a method of determining a relationship based on distance in parameter space is “nearest neighbor” analysis. Other non-limiting and illustrative methods are cluster analysis, self-organizing maps, and machine learning approaches. See generally, B. B. Ripley, Pattern Recognition and Neural Networks, Cambridge University Press, New York (1996).

These methods may be practiced in an iterative fashion, whereby the properties of lead peptides identified in a second test library are used to determine additional lead compounds in a third test library, etc., until compounds that provide the desired characteristics are identified. Moreover, the relationship determined in each iteration need not be fixed. One type of relationship may be chosen as identifying a set of second test peptides, but a different relationship may be chosen in subsequent iterations.

For example, indicia of an activity of a plurality of test peptides from a first test peptide library are measured. A relationship is then determined between at least one parameter and the measured indicia of the activity of the test peptides. Those skilled in the art will appreciate that the relationship may include “whole molecule” parameters (defined below) or sequence-specific parameters that vary with sequence. The relationship so identified is employed to determine a second test library containing a plurality of test peptides that are predicted to provide indicia of the growth inhibitory activity.

The first test compounds may be selected from a first test library of compounds (as were the peptides SEQ ID NO:1-SEQ ID NO:5) using a space-filling design. The first test compounds should be representative of the first test library. “Space-filling design” as used herein is intended to be construed broadly and include all such techniques known to those skilled in the art. Exemplary space-filling designs include full factorial designs, fractional factorial designs, maximum diversity libraries, genetic algorithms, coverage designs, spread designs, cluster based designs, Latin Hypercube Sampling, other optimal designs (e.g., D-Optimal), and the like. A space-filling design assists in selecting experimental design points. Space-filling designs provide a strategy for obtaining data at a set of design points, such that the data so obtained will efficiently represent all candidate compounds (the “candidate space”).

Any parameter (i.e., descriptor) known in the art that can be applied to characterize a compound may be used to carry out the present invention. Physical, chemical (including biochemical), biological and/or topological parameters may be employed to determine the relationship. The term “parameter” as used herein is also intended to encompass the principle components of Hellberg et al., supra. The parameter(s) used to describe the test compounds can change in both number and type during the selection process. In addition, the parameter(s) can be a whole molecule parameter(s), sequence specific parameter(s), or a combination of both.

Preferably, the compounds are characterized using at least one whole molecule parameter. A “whole molecule parameter” is a value that characterizes a molecule irrespective of the arrangement of its constitutive atoms. For example, a whole molecule parameter for a peptide is one that does not depend on the order or sequence of the amino acids. Describing a molecule using at least one whole molecule parameter facilitates the screening process by reducing (i.e., collapsing) the size of the compound space and thereby decreases the time, computational difficulty, and cost of screening large compound spaces. Conversely, a ‘sequence-specific” parameter is one that is dependent on the specific order or sequence of the constitutive atoms or subunits.

Illustrative parameters were described above. Most preferred herein are molecular weight, charge, total dipole moment, hydrophobicity (expressed as “Moriguchi logP” (mlogP or MlogP)). Calculations of parameters can be carried out by any method known in the art, for example, using a computerized system, e.g., a Silicon Graphics computer or a PC. Total charge, molecular weight, and total dipole can be calculated using the program Sybyl 6.5 (Tripos). MlogP can be calculated using a Sybyl Programming Language Script (as can calculations of the isoelectric point).

The relationship between the selected parameter or parameters of the growth inhibitory peptides disclosed herein and the measured indicia of the growth inhibitory property for each of the test compounds is used to identify a second plurality of useful peptides. Each of the second group of peptides may come from a second test library. The second test library could include all peptides that are predicted to satisfy the test requirement. Alternatively, and preferably, the second test library is chosen to include a subset thereof. The second set of test compounds may include all of the test compounds in the second test library or, alternatively, a subset thereof. For example, the second test library may include all peptides having five amino acids that are predicted to result in a certain inhibition of growth of a particular PDGF-R expressing cell line above a particular value (i.e.,, the test requirement) when added to culture medium in which the cells are grown. The second test compounds are preferably selected from and representative of the second test library—for example by using a space-filling design, as described

Derived using regression analysis, e.g., with the program S-Plus (Version 3.4 for 5 Solaris, Mathsoft, Seattle, Wash.), the following equation describes the relationship between three preferred parameters (hydrophobicity, molecular weight and charge) and the (hypothetical) indicia of the property (i.e., growth inhibition) mediated by a first set of test compounds (such as the four pentapeptides SEQ ID NO:2-SEQ ID NO:5):

ŷ=(3.64×MlogP)+(0.056×MW)−(1.97×charge)+1.73×R²=0.999 (1)

where ŷ is an estimated indicia of the property, MlogP is a measure of hydrophobicity, and MW is molecular weight. R² is a statistical measure of the amount of variability in the original response variable (ŷ) that is explained by the statistical model. An R² value of 0.999 specifies that 99.9% of the original variability in ŷ was explained by the statistical model.

If a satisfactory peptide (i.e., satisfies the test requirement) is not identified among a set of test peptides, the screening process continues. A second set of untested peptides can then be selected by any means known in the art, and the parameters for the second set of peptides may be calculated. Using Equation 1, the predicted activity of a second set of peptides can be calculated based on the parameters of the peptides included therein. This is exemplified in search for peptides with somewhat different biological activities Campbell et al., and Haaland et al., supra. A predicted activity derived for an untested peptide may exceed the growth inhibitory action of the five peptides noted above, rendering this new peptide a good candidate for synthesis and testing.

Values to describe the various parameters of the peptides, for example, hydrophobicity (i.e., MlogP), molecular weight, and total charge may be calculated for each peptide. Each peptide may be added to culture medium and growth of a selected type of cell or cell line or inhibition of growth (biological activity) may be measured for the cells cultured with each peptide. Real values for exemplary peptides taken from WO 01/07642 are shown in the table below to illustrate the analysis.

| | | mol. Wt | | Biological Activity |
|---|---|---|---|---|
| Peptide | Hydrophobicity | (Da) | Total Charge | (arbitrary units). |
| 1 | −3.479 | 469.5 | 0 | 15.0 |
| 2 | −1.608 | 486.5 | −1 | 25.0 |
| 3 | −3.479 | 501.5 | −1 | 19.3 |
| 4 | −3.421 | 416.4 | −1 | 14.4 |

Assume that there is a second set of untested (i.e., candidate) peptides with parameters as shown below:

| Peptide | Hydrophobicity | Mol. Wt | Total Charge | Biol. Act. |
|---|---|---|---|---|
| 5 | −4.03 | 496.5 | −2 | ? |
| 6 | −4.25 | 391.4 | −1 | ? |
| 7 | −1.278 | 474.5 | 0 | ? |
| 8 | −1.616 | 435.5 | −1 | ? |

The idea of the nearest neighbor rule is to find candidate peptides with parameters that are similar to those from the peptide with the “best” (in this case highest) observed biological activity or the “lead peptide.” Before performing any calculations, all parameters are typically standardized or normalized so that each will have an equal contribution to the nearest neighbor calculation. In this illustrative example, all parameters may be standardized so that they have values between 0 and 1. A standardized value may be computed in the following manner:

Standardized value=(Original value−Min. value)/(Max. value−Min. value) (2)

For above example the standardized value of molecular weight for Peptide 1 may be calculated as follows:

(469.5−391.4)/(501.5−391.4)=0.7092 (3)

The standardized parameter values for the eight peptides are displayed below

| Peptide | Hydrophobicity | Mol. Wt | Total Charge | Biol. Act. |
|---|---|---|---|---|
| 1 | 0.26 | 0.71 | 1 | 15.0 |
| 2 | 0.89 | 0.86 | 0.5 | 25.0 |
| 3 | 0.26 | 1 | 0.5 | 19.3 |
| 4 | 0.28 | 0.23 | 0.5 | 14.4 |
| 5 | 0.07 | 0.95 | 0 | ? |
| 6 | 0 | 0 | 0.5 | ? |
| 7 | 1 | 0.75 | 1 | ? |
| 8 | 0.89 | 0.40 | 0.5 | ? |

Once the standardized values have been calculated, nearest neighbors may be determined by calculating the Euclidean distances between the peptides in this 3-dimensional space (where 3 represents the number of parameters). For example, the distance between Peptide 1 and Peptide 7 is calculated as:

SQRT((0.26−1)²+(0.71−0.75)²+(1−1)²)=0.74

The table below shows these calculated distances between an initial (also referred to as “training”) set of 4 peptides. The peptides in the candidate set will then be assigned predicted indicia of the property based the closest peptide in the training set. The observed biological activities for these four peptides may then be measured as shown in this table (where arbitrary values are shown from a hypothetical experiment).

| Candidate | Closest | Predicted | Observed |
|---|---|---|---|
| Peptide | Peptide | Activity | Activity |
| 5 | 3 | 19.3 | 18.5 |
| 6 | 4 | 14.4 | 10.2 |
| 7 | 2 | 25.0 | 23.6 |
| 8 | 2 | 25.0 | 22.0 |

The test rule is to test candidate peptides that are similar to the best members from the first test library. Thus, in this example, Peptides 7 and 8 may be selected for synthesis and tested. If either or both of the peptides satisfy the test requirement, the screening process may be stopped at this point. Alternatively, if a peptide has not yet been identified, or if additional peptides are desired, the process can be continued in an iterative fashion. As a further alternative, the selection and screening process can be continued using a different relationship, e.g., a QSAR relationship as described above.

After the actual indicia of the property have been measured, the indicia (y-axis) for each peptide (x-axis) may be plotted in ascending (or conversely, in descending) order. Those compounds that satisfy the test requirement are selected as lead compounds and the parameter space surrounding some or all of these leads may be explored further.

In nearest neighbor analysis of a particular lead peptide, for illustrative purposes, two parameters (e.g., total dipole and hydrophobicity) may be employed. The standardized values (as described above) for the two parameters are plotted on the x- and y-axis. Concentric circles can be drawn through the parameter space to represent a particular cut-off in Euclidean distance from the lead peptide. In one embodiment, a space-filling design is used to find points in parameter space. The reason for extending the space around the lead peptide (concentric circles) is to gather information as to how close peptides must be in parameter space to exhibit similar activities, characteristics, or indicia of the property(ies) of interest.

A cut-off distance is established for each lead compound. If the data measured on the first group of test peptides are clustered, the cut-off distance will be smaller than if the data points are more dispersed. Once a cut-off distance has been determined, a second library of, for example, 5 second test compounds that fall within the cut-off space can be identified. The second test compounds are predicted to have activity that are similar to, or even better than, the closest lead compound. All or a subset of the second test compounds in the second test library are evaluated for activity. A space-filling design can be used to select for screening a subset of the entire second test library.

Relying on a second data set, a “final” most preferred compound may be identified or yet another set of lead compounds can be determined and used with nearest neighbor analysis (or some other approach) to identify a third set of peptides for screening. The screening process can be iterated as many times as necessary to identify peptides exhibiting suitable indicia of the property(ies).

The Examples below demonstrate how peptides with different characteristics were tested for bioactivity by their addition to cultured NIH3T3 cells that had been transfected with PDGF-ββ. These fibroblast-type cells overexpress the PDGF ββ homodimer, which remains tightly associated with the cell surface. NIH3T3-PDGF-ββ cells represent a model system that mimics the biological events involved in many types of cancer cells. These cells exhibit uncontrolled growth due to autocrine activation of PDGF-R by PDGF. This autocrine activation of cell growth was inhibited unexpectedly by the novel peptides described herein that exerted growth inhibitory effects when added to defined culture medium at concentrations above 3 mM.

As described above, the PDGF-R superfamily includes, in addition to PDGF-R the related kinases Flt and KDR. These molecules are involved in blood vessel formation and nourishment of solid tumors. By inhibiting PDGF-R and, preferably, one or more of these related tyrosine kinases, aberrant cell growth and the nutritional support for such growth in vivo are inhibited. The peptides of the present invention are successful at inhibiting one or more of these activities as demonstrated by studies in which the peptides inhibited growth of NIH3T3 cells overexpressing human PDGF-ββ, which is a well-accepted model for PDGF-R-dependent cancers.

While the results presented herein demonstrate the growth-inhibitory effect of the present peptides on a cell line which grows in a PDGF-R-dependent manner, (and which is an accepted model system for PDGF-R driven cancers), the use of these peptides in the treatment of cell proliferation disorders which are not PDGF-R driven are within the scope of the present invention.

Peptide Compositions

A preferred composition is, or comprises, a biologically active growth-inhibitory peptide as described herein characterized in that it binds to PDGF-R or otherwise inhibits PDGF-R or PDGF activity.

Moreover, a biologically active peptide has the relevant growth inhibitory activity, characterized, for example as the binding to PDGF-R and/or inhibition of growth of NIH3T3-PDGF-ββ cells in an in vitro or in vivo assay of binding or of cell growth. Preferably the peptide inhibits growth of these cells at a level at least about 20% of the activity of suramin.

A preferred peptide comprises a minimal amino acid sequence selected from the following group: KKKK (SEQ ID NO: 1), DDEEK (SEQ IS NO: 2), KLMSY (SEQ ID NO: 3), FFFKK (SEQ ID NO: 4) and FFHPV (SEQ ID NO: 5), or a combination of one or more of these peptides. An additional variant of such a peptide has between 1-4 additional amino acids. Longer peptide multimers of the invention are described below.

Also included herein are compositions and methods using peptides with sequences that represent all possible permutations of SEQ ID NO:2-SEQ ID NO:5, inclusive (also termed “shuffled sequences”). See, for example, the table below listing shuffled sequences along side the “parent” sequences.

| Parent | Shuffled sequences |
|---|---|
| DDEEK | KEEDD (SEQ ID NO: 342), KEDDE (SEQ ID NO: 343), |
| SEQ ID | EEDDK (SEQ ID NO: 344), KEDED (SEQ ID NO: 345), |
| NO: 2) | KDDEE (SEQ ID NO: 346), EDDEK (SEQ ID NO: 347), |
| | EEDKD (SEQ ID NO: 348), EDDKE (SEQ ID NO: 349), |
| | KDEDE (SEQ ID NO: 350), EDEDK (SEQ ID NO: 351), |
| | KDEED (SEQ ID NO: 352), EDEKD (SEQ ID NO: 353), |
| | EEKDD (SEQ ID NO: 354), EDKDE (SEQ ID NO: 355), |
| | EDKED (SEQ ID NO: 356), DDKEE (SEQ ID NO: 357), |
| | DDEKE (SEQ ID NO: 358), EKEDD (SEQ ID NO: 359), |
| | EKDDE (SEQ ID NO: 360), EKDED (SEQ ID NO: 361), |
| | DKDEE (SEQ ID NO: 362), DEDEK (SEQ ID NO: 363), |
| | DEDKE (SEQ ID NO: 364), DKEDE (SEQ ID NO: 365), |
| | DEEDK (SEQ ID NO: 366), DKEED (SEQ ID NO: 367), |
| | DEEKD (SEQ ID NO: 368), DEKDE (SEQ ID NO: 369), |
| | DEKED (SEQ ID NO: 370) |
| KLMSY | YSMLK (SEQ ID NO: 371), YSMKL (SEQ ID NO: 372), |
| (SEQ ID | YSLKM (SEQ ID NO: 373), YMLKS (SEQ ID NO: 374), |
| NO: 3) | SMLKY (SEQ ID NO: 375), YSLMK (SEQ ID NO: 376), |
| | YSKML (SEQ ID NO: 377), YSKLM (SEQ ID NO: 378), |
| | YMKLS (SEQ ID NO: 379), SMKLY (SEQ ID NO: 380), |
| | YMLSK (SEQ ID NO: 381), YMKSL (SEQ ID NO: 382), |
| | YLKSM (SEQ ID NO: 383), YLKMS (SEQ ID NO: 384), |
| | SLKMY (SEQ ID NO: 385), SMLYK (SEQ ID NO: 386), |
| | SMKYL (SEQ ID NO: 387), SLKYM (SEQ ID NO: 388), |
| | MLKYS (SEQ ID NO: 389), MLKSY (SEQ ID NO: 390), |
| | YMSLK (SEQ ID NO: 391), YMSKL (SEQ ID NO: 392), |
| | YLSKM (SEQ ID NO: 393), YLMKS (SEQ ID NO: 394), |
| | SLMKY (SEQ ID NO: 395), YLSMK (SEQ ID NO: 396), |
| | YKSML (SEQ ID NO: 397), YKSLM (SEQ ID NO: 398), |
| | YKMLS (SEQ ID NO: 399), SKMLY (SEQ ID NO: 400), |
| | YLMSK (SEQ ID NO: 401), YKMSL (SEQ ID NO: 402), |
| | YKLSM (SEQ ID NO: 403), YKLMS (SEQ ID NO: 404), |
| | SKLMY (SEQ ID NO: 405), SLMYK (SEQ ID NO: 406), |
| | SKMYL (SEQ ID NO: 407), SKLYM (SEQ ID NO: 408), |
| | MKLYS (SEQ ID NO: 409), MKLSY (SEQ ID NO: 410), |
| | SMYLK (SEQ ID NO: 411), SMYKL (SEQ ID NO: 412), |
| | SLYKM (SEQ ID NO: 413), MLYKS (SEQ ID NO: 414), |
| | MLSKY (SEQ ID NO: 415), SLYMK (SEQ ID NO: 416), |
| | SKYML (SEQ ID NO: 417), SKYLM (SEQ ID NO: 418), |
| | MKYLS (SEQ ID NO: 419), MKSLY (SEQ ID NO: 420), |
| | MLYSK (SEQ ID NO: 421), MKYSL (SEQ ID NO: 422), |
| | LKYSM (SEQ ID NO: 423), LKYMS (SEQ ID NO: 424), |
| | LKSMY (SEQ ID NO: 425), MLSYK (SEQ ID NO: 426), |
| | MKSYL (SEQ ID NO: 427), LKSYM (SEQ ID NO: 428), |
| | LKMYS (SEQ ID NO: 429), LKMSY (SEQ ID NO: 430), |
| | SYMLK (SEQ ID NO: 431), SYMKL (SEQ ID NO: 432), |
| | SYLKM (SEQ ID NO: 433), MYLKS (SEQ ID NO: 434), |
| | MSLKY (SEQ ID NO: 435), SYLMK (SEQ ID NO: 436), |
| | SYKML (SEQ ID NO: 437), SYKLM (SEQ ID NO: 438), |
| | MYKLS (SEQ ID NO: 439), MSKLY (SEQ ID NO: 440), |
| | MYLSK (SEQ ID NO: 441), MYKSL (SEQ ID NO: 442), |
| | LYKSM (SEQ ID NO: 443), LYKMS (SEQ ID NO: 444), |
| | LSKMY (SEQ ID NO: 445), MSLYK (SEQ ID NO: 446), |
| | MSKYL (SEQ ID NO: 447), LSKYM (SEQ ID NO: 448), |
| | LMKYS (SEQ ID NO: 449), LMKSY (SEQ ID NO: 450), |
| | MYSLK (SEQ ID NO: 451), MYSKL (SEQ ID NO: 452), |
| | LYSKM (SEQ ID NO: 453), LYMKS (SEQ ID NO: 454), |
| | LSMKY (SEQ ID NO: 455), LYSMK (SEQ ID NO: 456), |
| | KYSML (SEQ ID NO: 457), KYSLM (SEQ ID NO: 458), |
| | KYMLS (SEQ ID NO: 459), KSMLY (SEQ ID NO: 460), |
| | LYMSK (SEQ ID NO: 461), KYMSL (SEQ ID NO: 462), |
| | KYLSM (SEQ ID NO: 463), KYLMS (SEQ ID NO: 464), |
| | KSLMY (SEQ ID NO: 465), LSMYK (SEQ ID NO: 466), |
| | KSMYL (SEQ ID NO: 467), KSLYM (SEQ ID NO: 468), |
| | KMLYS (SEQ ID NO: 469), KMLSY (SEQ ID NO: 470), |
| | MSYLK (SEQ ID NO: 471), MSYKL (SEQ ID NO: 472), |
| | LSYKM (SEQ ID NO: 473), LMYKS (SEQ ID NO: 474), |
| | LMSKY (SEQ ID NO: 475), LSYMK (SEQ ID NO: 476), |
| | KSYML (SEQ ID NO: 477), KSYLM (SEQ ID NO: 478), |
| | KMYLS (SEQ ID NO: 479), KMSLY (SEQ ID NO: 480), |
| | LMYSK (SEQ ID NO: 481), KMYSL (SEQ ID NO: 482), |
| | KLYSM (SEQ ID NO: 483), KLYMS (SEQ ID NO: 484), |
| | KLSMY (SEQ ID NO: 485), LMSYK (SEQ ID NO: 486), |
| | KMSYL (SEQ ID NO: 487), KLSYM (SEQ ID NO: 488), |
| | KLMYS (SEQ ID NO: 489). |
| FFFKK | KKFFF (SEQ ID NO: 490), KFFFK (SEQ ID NO: 491), |
| (SEQ ID | KFFKF (SEQ ID NO: 492), KFKFF (SEQ ID NO: 493), |
| NO:4) | FFKFK (SEQ ID NO: 494), FFKKF (SEQ ID NO: 495), |
| | FKFFK (SEQ ID NO: 496), FKFKF (SEQ ID NO: 497), |
| | FKKFF (SEQ ID NO: 498) |
| FFHPV | VPHFF (SEQ ID NO: 499), VPFFH (SEQ ID NO: 500), |
| (SEQ ID | VHFFP (SEQ ID NO: 501), PHFFV (SEQ ID NO: 502), |
| NO:5) | VPFHF (SEQ ID NO: 503), VHFPF (SEQ ID NO: 504), |
| | VFFPH (SEQ ID NO: 505), VFFHP (SEQ ID NO: 506), |
| | PFFHV (SEQ ID NO: 507), PHFVF (SEQ ID NO: 508), |
| | PFFVH (SEQ ID NO: 509), HFFVP (SEQ ID NO: 510), |
| | HFFPV (SEQ ID NO: 511), VHPFF (SEQ ID NO: 512), |
| | VFPFH (SEQ ID NO: 513), VFHFP (SEQ ID NO: 514), |
| | PFHFV (SEQ ID NO: 515), VFPHF (SEQ ID NO: 516), |
| | VFHPF (SEQ ID NO: 517), PFHVF (SEQ ID NO: 518), |
| | PHVFF (SEQ ID NO: 519), PFVFH (SEQ ID NO: 520), |
| | HFVFP (SEQ ID NO: 521), HFPFV (SEQ ID NO: 522), |
| | PFVHF (SEQ ID NO: 523), HFVPF (SEQ ID NO: 524), |
| | FFVPH (SEQ ID NO: 525), FFVHP (SEQ ID NO: 526), |
| | FFPHV (SEQ ID NO: 527), HFPVF (SEQ ID NO: 528), |
| | FFPVH (SEQ ID NO: 529), FFHVP (SEQ ID NO: 530), |
| | PVHFF (SEQ ID NO: 531), PVFFH (SEQ ID NO: 532), |
| | HVFFP (SEQ ID NO: 533), HPFFV (SEQ ID NO: 534), |
| | PVFHF (SEQ ID NO: 535), HVFPF (SEQ ID NO: 536), |
| | FVFPH (SEQ ID NO: 537), FVFHP (SEQ ID NO: 538), |
| | FPFHV (SEQ ID NO: 539), HPFVF (SEQ ID NO: 540), |
| | FPFVH (SEQ ID NO: 541), FHFVP (SEQ ID NO: 542), |
| | FHFPV (SEQ ID NO: 543), HVPFF (SEQ ID NO: 544), |
| | FVPFH (SEQ ID NO: 545), FVHFP (SEQ ID NO: 546), |
| | FPHFV (SEQ ID NO: 547), FVPHF (SEQ ID NO: 548), |
| | FVHPF (SEQ ID NO: 549), FPHVF (SEQ ID NO: 550), |
| | HPVFF (SEQ ID NO: 551), FPVFH (SEQ ID NO: 552), |
| | FHVFP (SEQ ID NO: 553), FHPFV (SEQ ID NO: 554), |
| | FPVHF (SEQ ID NO: 555), FHVPF (SEQ ID NO: 556), |
| | FHPVF (SEQ ID NO: 557) |

The peptide may be capped at its N and C termini with an acyl (abbreviated “Ac”)—and an amido (abbreviated “Am”) group, respectively, for example acetyl (CH₃CO—) at the N terminus and amido (—NH₂) at the C terminus. Capping increases stability in vivo.

A broad range of N-terminal capping functions, preferably in a linkage to the terminal amino group, is contemplated, for example:

formyl;

alkanoyl, having from 1 to 10 carbon atoms, such as acetyl, propionyl, butyryl;

alkenoyl, having from 1 to 10 carbon atoms, such as hex-3-enoyl;

alkynoyl, having from 1 to 10 carbon atoms, such as hex-5-ynoyl;

aroyl, such as benzoyl or 1-naphthoyl;

heteroaroyl, such as 3-pyrroyl or 4-quinoloyl;

alkylsulfonyl, such as methanesulfonyl;

arylsulfonyl, such as benzenesulfonyl or sulfanilyl;

heteroarylsulfonyl, such as pyridine-4-sulfonyl;

substituted alkanoyl, having from 1 to 10 carbon atoms, such as 4-aminobutyryl;

substituted alkenoyl, having from 1 to 10 carbon atoms, such as 6-hydroxy-hex-3-enoyl;

substituted alkynoyl, having from 1 to 10 carbon atoms, such as 3-hydroxy-hex-5-ynoyl;

substituted aroyl, such as 4-chlorobenzoyl or 8-hydroxy-naphth-2-oyl;

substituted heteroaroyl, such as 2,4-dioxo-1,2,3,4-tetrahydro-3-methyl-quinazolin-6-oyl;

substituted alkylsulfonyl, such as 2-aminoethanesulfonyl;

substituted arylsulfonyl, such as 5-dimethylamino-1-naphthalenesulfonyl;

substituted heteroarylsulfonyl, such as 1-methoxy-6-isoquinolinesulfonyl;

carbamoyl or thiocarbamoyl;

substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl;

substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

The C-terminal capping function can either be in an amide or ester bond with the terminal carboxyl. Capping functions that provide for an amide bond are designated as NR¹R² wherein R¹ and R² may be independently drawn from the following group:

hydrogen;

alkyl, preferably having from 1 to 10 carbon atoms, such as methyl, ethyl, isopropyl;

alkenyl, preferably having from 1 to 10 carbon atoms, such as prop-2-enyl;

alkynyl, preferably having from 1 to 10 carbon atoms, such as prop-2-ynyl;

substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;

substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;

substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;

aroylalkyl having up to 10 carbon atoms, such as phenacyl or 2-benzoylethyl;

aryl, such as phenyl or 1-naphthyl;

heteroaryl, such as 4-quinolyl;

alkanoyl having from 1 to 10 carbon atoms, such as acetyl or butyryl;

aroyl, such as benzoyl;

heteroaroyl, such as 3-quinoloyl;

OR′ or NR′R″ where R′ and R″ are independently hydrogen, alkyl, aryl, heteroaryl, acyl, aroyl, sulfonyl, sulfinyl, or SO₂—R′″ or SO—R′″ where R′″ is substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

Either the N-terminal or the C-terminal capping function, or both, may be of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules.

Production of Peptides and Derivatives

General Chemical Synthetic Procedures

The peptides of the invention may be prepared using recombinant DNA technology. However, given their length, they are preferably prepared using solid-phase synthesis, such as that generally described by Merrifield, J. Amer. Chem. Soc., 85:2149-54 (1963), although other equivalent chemical syntheses known in the art are also useful, for example, the FMOC chemistry of Atherton and Sheppard, 1989 (In: Solid-Phase Peptide Synthesis: A Practical Approach, E. Atherton and R. C. Sheppard; Oxford University Press; Oxford, 1989). t-Boc chemistry may also be used as well as synthesis on a variety of different solid supports, “tea-bag” synthesis (See, Pinilla, C et al., Meth. Molec. Biol., 66:171-179 (1996)), and split and divide combinatorial methods. Solid-phase peptide synthesis may be initiated from the C-terminus of the peptide by coupling a protected α-amino acid to a suitable resin. Such a starting material can be prepared by attaching an α-amino-protected amino acid by an ester linkage to a chloromethylated resin or to a hydroxymethyl resin, or by an amide bond to a BHA resin or MBHA resin. Such methods, well known in the art, are disclosed, for example, in U.S. Pat. No. 5,994,309, which is incorporated by reference in its entirety. Solution phase methods for peptide synthesis may also be used.

As an alternative to chemical or enzymatic synthesis, the peptides of the present invention may be produced using recombinant methods. For recombinant production, a nucleic acid sequence encoding the desired peptide sequence is determined. This may be an RNA sequence that is subsequently translated to produce the peptide, or a DNA sequence that is then cloned into an expression vector under the control of a promoter that enables the transcription of the DNA sequence and subsequence translation of the mRNA to produce the peptide.

For example, short single-stranded DNA fragments may be prepared by the phosphoramidite method (Beaucage et al., Tetrahed. Lett., 22: 1859-1862 (1981)). A double-stranded fragment then may be obtained either by synthesizing the complementary strand and annealing the strands together under appropriate conditions or by adding the complementary strand using DNA polymerase with an appropriate primer sequence. DNA fragments encoding the peptide will be incorporated in DNA constructs capable of introduction to and expression in cells in culture.

Preferred nucleic acid molecules of the present invention are those that encode the inhibitory peptides, preferably any one of more of SEQ ID NO:1 through SEQ ID NO:5, inclusive. The following nucleic acid sequences (SEQ ID NO:7-SEQ ID NO:341, inclusive) and DNA or RNA molecules that include one of more of these following sequences are within the scope of this invention. These may be used in the production of recombinant polypeptides or as means for expressing polypeptides in cells in vitro or in vivo.

(1) Nucleotide sequences encoding Lys Lys Lys Lys (SEQ ID NO:1):

| AAA AAA AAA AAA | (SEQ ID NO:6) | | AAG AAA AAA AAA | (SEQ ID NO:14) |
|---|---|---|---|---|
| AAA AAA AAA AAG | (SEQ ID NO:7) | | AAG AAA AAA AAG | (SEQ ID NO:15) |
| AAA AAA AAG AAA | (SEQ ID NO:8) | 20 | AAG AAA AAG AAA | (SEQ ID NO:16) |
| AAA AAA AAG AAG | (SEQ ID NO:9) | | AAG AAA AAG AAG | (SEQ ID NO:17) |
| AAA AAG AAA AAA | (SEQ ID NO:10) | | AAG AAG AAA AAA | (SEQ ID NO:18) |
| AAA AAG AAA AAG | (SEQ ID NO:11) | | AAG AAG AAA AAG | (SEQ ID NO:19) |
| AAA AAG AAG AAA | (SEQ ID NO:12) | | AAG AAG AAG AAA | (SEQ ID NO:20) |
| AAA AAG AAG AAG | (SEQ ID NO:13) | 25 | AAG AAG AAG AAG | (SEQ ID NO:21) |

(2) Nucleotide sequences encoding ASP ASP GLU GLU LYS (SEQ ID NO:2)

| | | | | |
|---|---|---|---|---|
| GAT GAT GAA GAA AAA | (SEQ ID NO:22) | 45 | GAT GAC GAA GAA AAA | (SEQ ID NO:38) |
| GAT GAT GAA GAG AAA | (SEQ ID NO:23) | | GAT GAC GAA GAG AAA | (SEQ ID NO:39) |
| GAT GAT GAG GAG AAA | (SEQ ID NO:24) | | GAT GAC GAG GAA AAA | (SEQ ID NO:40) |
| GAT GAT GAG GAA AAA | (SEQ ID NO:25) | | GAT GAC GAG GAG AAA | (SEQ ID NO:41) |
| GAT GAT GAA GAA AAG | (SEQ ID NO:26) | | GAT GAC GAA GAA AAG | (SEQ ID NO:42) |
| GAT GAT GAG GAG AAG | (SEQ ID NO:27) | 50 | GAT GAC GAA GAG AAG | (SEQ ID NO:43) |
| GAT GAT GAG GAA AAG | (SEQ ID NO:28) | | GAT GAC GAG GAA AAG | (SEQ ID NO:44) |
| GAT GAT GAA GAG AAG | (SEQ ID NO:29) | | GAT GAC GAG GAG AAG | (SEQ ID NO:45) |
| GAC GAC GAA GAA AAA | (SEQ ID NO:30) | | GAC GAT GAA GAA AAA | (SEQ ID NO:46) |
| GAC GAC GAG GAA AAA | (SEQ ID NO:31) | | GAC GAT GAA GAG AAA | (SEQ ID NO:47) |
| GAC GAC GAG GAG AAA | (SEQ ID NO:32) | 55 | GAC GAT GAG GAA AAA | (SEQ ID NO:48) |
| GAC GAC GAA GAG AAA | (SEQ ID NO:33) | | GAC GAT GAG GAG AAA | (SEQ ID NO:49) |
| GAC GAC GAA GAA AAG | (SEQ ID NO:34) | | GAC GAT GAA GAA AAG | (SEQ ID NO:50) |
| GAC GAC GAG GAA AAG | (SEQ ID NO:35) | | GAC GAT GAA GAG AAG | (SEQ ID NO:51) |
| GAC GAC GAA GAG AAG | (SEQ ID NO:36) | | GAC GAT GAG GAA AAG | (SEQ ID NO:52) |
| GAC GAC GAG GAG AAG | (SEQ ID NO:37) | 60 | GAG GAT GAG GAG AAG | (SEQ ID NO:53) |

(3) Nucleotide sequences encoding LYS LEU MET SER TYR (SEQ ID NO:3)

| | | | | |
|---|---|---|---|---|
| AAA CTT ATA TCT TAT | (SEQ ID NO:54) | | AAA CTT ATA TCA TAT | (SEQ ID NO:58) |
| AAA CTT ATA TCT TAC | (SEQ ID NO:55) | | AAA CTT ATA TCA TAC | (SEQ ID NO:59) |
| AAA CTT ATA TCC TAT | (SEQ ID NO:56) | 70 | AAA CTT ATA TCG TAT | (SEQ ID NO:60) |
| AAA CTT ATA TCC TAC | (SEQ ID NO:57) | | AAA CTT ATA TCG TAC | (SEQ ID NO:61) |
| AAA CTT ATG TCT TAT | (SEQ ID NO:62) | | AAA CTG ATG TCC TAC | (SEQ ID NO:113) |
| AAA CTT ATG TCT TAC | (SEQ ID NO:63) | | AAA CTG ATG TCA TAT | (SEQ ID NO:114) |
| AAA CTT ATG TCC TAT | (SEQ ID NO:64) | | AAA CTG ATG TCA TAC | (SEQ ID NO:115) |
| AAA CTT ATG TCC TAC | (SEQ ID NO:65) | 55 | AAA CTG ATG TCG TAT | (SEQ ID NO:116) |
| AAA CTT ATG TCA TAT | (SEQ ID NO:66) | | AAA CTG ATC TCG TAC | (SEQ ID NO:117) |
| AAA CTT ATG TCA TAC | (SEQ ID NO:67) | | AAG CTT ATA TCT TAT | (SEQ ID NO:118) |
| AAA CTT ATG TCG TAT | (SEQ ID NO:68) | | AAG CTT ATA TCT TAC | (SEQ ID NO:119) |
| AAA CTT ATC TCG TAC | (SEQ ID NO:69) | | AAG CTT ATA TCC TAT | (SEQ ID NO:120) |
| AAA CTC ATA TCT TAT | (SEQ ID NO:70) | 60 | AAG CTT ATA TCC TAC | (SEQ ID NO:121) |
| AAA CTC ATA TCT TAC | (SEQ ID NO:71) | | AAG CTT ATA TCA TAT | (SEQ ID NO:122) |
| AAA CTC ATA TCC TAT | (SEQ ID NO:72) | | AAG CTT ATA TCA TAC | (SEQ ID NO:123) |
| AAA CTC ATA TCC TAC | (SEQ ID NO:73) | | AAG CTT ATA TCG TAT | (SEQ ID NO:124) |
| AAA CTC ATA TCA TAT | (SEQ ID NO:74) | | AAG CTT ATA TCG TAC | (SEQ ID NO:125) |
| AAA CTC ATA TCA TAC | (SEQ ID NO:75) | 65 | AAG CTT ATG TCT TAT | (SEQ ID NO:126) |
| AAA CTC ATA TCG TAT | (SEQ ID NO:76) | | AAG CTT ATG TCT TAC | (SEQ ID NO:127) |
| AAA CTC ATA TCG TAC | (SEQ ID NO:77) | | AAG CTT ATG TCC TAT | (SEQ ID NO:128) |
| AAA CTC ATG TCT TAT | (SEQ ID NO:78) | | AAG CTT ATG TCC TAC | (SEQ ID NO:129) |
| AAA CTC ATG TCT TAC | (SEQ ID NO:79) | | AAG CTT ATG TCA TAT | (SEQ ID NO:130) |
| AAA CTC ATG TCC TAT | (SEQ ID NO:80) | 70 | AAG CTT ATG TCA TAC | (SEQ ID NO:131) |
| AAA CTC ATG TCC TAC | (SEQ ID NO:81) | | AAG CTT ATG TCG TAT | (SEQ ID NO:132) |
| AAA CTC ATG TCA TAT | (SEQ ID NO:82) | | AAG CTT ATC TCG TAC | (SEQ ID NO:133) |
| AAA CTC ATG TCA TAC | (SEQ ID NO:83) | | AAG CTC ATA TCT TAT | (SEQ ID NO:134) |
| AAA CTC ATG TCG TAT | (SEQ ID NO:84) | | AAG CTC ATA TCT TAC | (SEQ ID NO:135) |
| AAA CTC ATC TCG TAC | (SEQ ID NO:85) | 75 | AAG CTC ATA TCC TAT | (SEQ ID NO:136) |
| AAA CTA ATA TCT TAT | (SEQ ID NO:86) | | AAG CTC ATA TCC TAC | (SEQ ID NO:137) |
| AAA CTA ATA TCT TAC | (SEQ ID NO:87) | | AAG CTC ATA TCA TAT | (SEQ ID NO:138) |
| AAA CTA ATA TCC TAT | (SEQ ID NO:88) | | AAG CTC ATA TCA TAC | (SEQ ID NO:139) |
| AAA CTA ATA TCC TAC | (SEQ ID NO:89) | | AAG CTC ATA TCG TAT | (SEQ ID NO:140) |
| AAA CTA ATA TCA TAT | (SEQ ID NO:90) | 80 | AAG CTC ATA TCG TAC | (SEQ ID NO:141) |
| AAA CTA ATA TCA TAC | (SEQ ID NO:91) | | AAG CTC ATG TCT TAT | (SEQ ID NO:142) |
| AAA CTA ATA TCG TAT | (SEQ ID NO:92) | | AAG CTC ATG TCT TAC | (SEQ ID NO:143) |
| AAA CTA ATA TCG TAC | (SEQ ID NO:93) | | AAG CTC ATG TCC TAT | (SEQ ID NO:144) |
| AAA CTA ATG TCT TAT | (SEQ ID NO:94) | | AAG CTC ATG TCC TAC | (SEQ ID NO:145) |
| AAA CTA ATG TCT TAC | (SEQ ID NO:95) | 85 | AAG CTC ATG TCA TAT | (SEQ ID NO:146) |
| AAA CTA ATG TCC TAT | (SEQ ID NO:96) | | AAG CTC ATG TCA TAC | (SEQ ID NO:147) |
| AAA CTA ATG TCC TAC | (SEQ ID NO:97) | | AAG CTC ATG TCG TAT | (SEQ ID NO:148) |
| AAA CTA ATG TCA TAT | (SEQ ID NO:98) | | AAG CTC ATC TCG TAC | (SEQ ID NO:149) |
| AAA CTA ATG TCA TAC | (SEQ ID NO:99) | | AAG CTA ATA TCT TAT | (SEQ ID NO:150) |
| AAA CTA ATG TCG TAT | (SEQ ID NO:100) | 90 | AAG CTA ATA TCT TAC | (SEQ ID NO:151) |
| AAA CTG ATC TCG TAC | (SEQ ID NO:101) | | AAG CTA ATA TCC TAT | (SEQ ID NO:152) |
| AAA CTG ATA TCT TAT | (SEQ ID NO:102) | | AAG CTA ATA TCC TAC | (SEQ ID NO:153) |
| AAA CTG ATA TCT TAC | (SEQ ID NO:103) | | AAG CTA ATA TCA TAT | (SEQ ID NO:154) |
| AAA CTG ATA TCC TAT | (SEQ ID NO:104) | | AAG CTA ATA TCA TAC | (SEQ ID NO:155) |
| AAA CTG ATA TCC TAC | (SEQ ID NO:105) | 95 | AAG CTA ATA TCG TAT | (SEQ ID NO:156) |
| AAA CTG ATA TCA TAT | (SEQ ID NO:106) | | AAG CTA ATA TCG TAC | (SEQ ID NO:157) |
| AAA CTG ATA TCA TAC | (SEQ ID NO:107) | | AAG CTA ATG TCT TAT | (SEQ ID NO:158) |
| AAA CTG ATA TCG TAT | (SEQ ID NO:108) | | AAG CTA ATG TCT TAC | (SEQ ID NO:159) |
| AAA CTG ATA TCG TAC | (SEQ ID NO:109) | | AAG CTA ATG TCC TAT | (SEQ ID NO:160) |
| AAA CTG ATG TCT TAT | (SEQ ID NO:110) | 100 | AAG CTA ATG TCC TAC | (SEQ ID NO:161) |
| AAA CTG ATG TCT TAC | (SEQ ID NO:111) | | AAG CTA ATG TCA TAT | (SEQ ID NO:162) |
| AAA CTG ATG TCC TAT | (SEQ ID NO:112) | | AAG CTA ATG TCA TAC | (SEQ ID NO:163) |
| AAG CTA ATG TCG TAT | (SEQ ID NO:164) | 10 | AAG CTG ATA TCG TAC | (SEQ ID NO:173) |
| AAG CTG ATC TCG TAC | (SEQ ID NO:165) | | AAG CTG ATG TCT TAT | (SEQ ID NO:174) |
| AAG CTG ATA TCT TAT | (SEQ ID NO:166) | | AAG CTG ATG TCT TAC | (SEQ ID NO:175) |
| AAG CTG ATA TCT TAC | (SEQ ID NO:167) | | AAG CTG ATG TCC TAT | (SEQ ID NO:176) |
| AAG CTG ATA TCC TAT | (SEQ ID NO:168) | | AAG CTG ATG TCC TAC | (SEQ ID NO:177) |
| AAG CTG ATA TCC TAC | (SEQ ID NO:169) | 15 | AAG CTG ATG TCA TAT | (SEQ ID NO:178) |
| AAG CTG ATA TCA TAT | (SEQ ID NO:170) | | AAG CTG ATG TCA TAC | (SEQ ID NO:179) |
| AAG CTG ATA TCA TAC | (SEQ ID NO:171) | | AAG CTG ATG TCG TAT | (SEQ ID NO:180) |
| AAG CTG ATA TCG TAT | (SEQ ID NO:172) | | AAG CTG ATC TCG TAC | (SEQ ID NO:181) |

(4) Nucleotide sequences encoding PHE PHE PHE LYS LYS (SEQ ID NO:4):

| | | | | |
|---|---|---|---|---|
| TTT TTT TTT AAA AAA | (SEQ ID NO:182) | | TTC TTT TTT AAA AAA | (SEQ ID NO:198) |
| TTT TTT TTT AAA AAG | (SEQ ID NO:183) | | TTC TTT TTT AAA AAG | (SEQ ID NO:199) |
| TTT TTT TTT AAG AAA | (SEQ ID NO:184) | | TTC TTT TTT AAG AAA | (SEQ ID NO:200) |
| TTT TTT TTT AAG AAG | (SEQ ID NO:185) | 40 | TTC TTT TTT AAG AAG | (SEQ ID NO:201) |
| TTT TTT TTC AAA AAA | (SEQ ID NO:186) | | TTC TTT TTC AAA AAA | (SEQ ID NO:202) |
| TTT TTT TTC AAA AAG | (SEQ ID NO:187) | | TTC TTT TTC AAA AAG | (SEQ ID NO:203) |
| TTT TTT TTC AAG AAA | (SEQ ID NO:188) | | TTC TTT TTC AAG AAA | (SEQ ID NO:204) |
| TTT TTT TTC AAG AAG | (SEQ ID NO:189) | | TTC TTT TTC AAG AAG | (SEQ ID NO:205) |
| TTT TTC TTT AAA AAA | (SEQ ID NO:190) | 45 | TTC TTC TTT AAA AAA | (SEQ ID NO:206) |
| TTT TTC TTT AAA AAG | (SEQ ID NO:191) | | TTC TTC TTT AAA AAG | (SEQ ID NO:207) |
| TTT TTC TTT AAG AAA | (SEQ ID NO:192) | | TTC TTC TTT AAG AAA | (SEQ ID NO:208) |
| TTT TTC TTT AAG AAG | (SEQ ID NO:193) | | TTC TTC TTT AAG AAG | (SEQ ID NO:209) |
| TTT TTC TTC AAA AAA | (SEQ ID NO:194) | | TTC TTC TTC AAA AAA | (SEQ ID NO:210) |
| TTT TTC TTC AAA AAG | (SEQ ID NO:195) | 50 | TTC TTC TTC AAA AAG | (SEQ ID NO:211) |
| TTT TTC TTC AAG AAA | (SEQ ID NO:196) | | TTC TTC TTC AAG AAA | (SEQ ID NO:212) |
| TTT TTC TTC AAG AAG | (SEQ ID NO:197) | | TTC TTC TTC AAG AAG | (SEQ ID NO:213) |

(5) Nucleotide sequences encoding PHE PHE HIS PRO VAL (SEQ ID NO:5)

| | | | | |
|---|---|---|---|---|
| TTT TTT CAT CCT GTT | (SEQ ID NO:214) | 75 | TTT TTT CAC CCT GTG | (SEQ ID NO:233) |
| TTT TTT CAT CCT GTC | (SEQ ID NO:215) | | TTT TTT CAC CCC GTT | (SEQ ID NO:234) |
| TTT TTT CAT CCT GTA | (SEQ ID NO:216) | | TTT TTT CAC CCC GTC | (SEQ ID NO:235) |
| TTT TTT CAT CCT GTG | (SEQ ID NO:217) | | TTT TTT CAC CCC GTA | (SEQ ID NO:236) |
| TTT TTT CAT CCC GTT | (SEQ ID NO:218) | | TTT TTT CAC CCC GTG | (SEQ ID NO:237) |
| TTT TTT CAT CCC GTC | (SEQ ID NO:219) | 80 | TTT TTT CAC CCA GTT | (SEQ ID NO:238) |
| TTT TTT CAT CCC GTA | (SEQ ID NO:220) | | TTT TTT CAC CCA GTC | (SEQ ID NO:239) |
| TTT TTT CAT CCC GTG | (SEQ ID NO:221) | | TTT TTT CAC CCA GTA | (SEQ ID NO:240) |
| TTT TTT CAT CCA GTT | (SEQ ID NO:222) | | TTT TTT CAC CCA GTG | (SEQ ID NO:241) |
| TTT TTT CAT CCA GTC | (SEQ ID NO:223) | | TTT TTT CAC CCG GTT | (SEQ ID NO:242) |
| TTT TTT CAT CCA GTA | (SEQ ID NO:224) | 85 | TTT TTT CAC CCG GTC | (SEQ ID NO:243) |
| TTT TTT CAT CCA GTG | (SEQ ID NO:225) | | TTT TTT CAC CCG GTA | (SEQ ID NO:244) |
| TTT TTT CAT CCG GTT | (SEQ ID NO:226) | | TTT TTT CAC CCG GTG | (SEQ ID NO:245) |
| TTT TTT CAT CCG GTC | (SEQ ID NO:227) | | TTT TTC CAT CCT GTT | (SEQ ID NO:246) |
| TTT TTT CAT CCG GTA | (SEQ ID NO:228) | | TTT TTC CAT CCT GTC | (SEQ ID NO:247) |
| TTT TTT CAT CCG GTG | (SEQ ID NO:229) | 90 | TTT TTC CAT CCT GTA | (SEQ ID NO:248) |
| TTT TTT CAC CCT GTT | (SEQ ID NO:230) | | TTT TTC CAT CCT GTG | (SEQ ID NO:249) |
| TTT TTT CAC CCT GTC | (SEQ ID NO:231) | | TTT TTC CAT CCC GTT | (SEQ ID NO:250) |
| TTT TTT CAC CCT GTA | (SEQ ID NO:232) | | TTT TTC CAT CCC GTC | (SEQ ID NO:251) |
| TTT TTC CAT CCC GTA | (SEQ ID NO:252) | | TTC TTT CAC CCT GTG | (SEQ ID NO:297) |
| TTT TTC CAT CCC GTG | (SEQ ID NO:253) | | TTC TTT CAC CCC GTT | (SEQ ID NO:298) |
| TTT TTC CAT CCA GTT | (SEQ ID NO:254) | | TTC TTT CAC CCC GTC | (SEQ ID NO:299) |
| TTT TTC CAT CCA GTC | (SEQ ID NO:255) | | TTC TTT CAC CCC GTA | (SEQ ID NO:300) |
| TTT TTC CAT CCA GTA | (SEQ ID NO:256) | 50 | TTC TTT CAC CCC GTG | (SEQ ID NO:301) |
| TTT TTC CAT CCA GTG | (SEQ ID NO:257) | | TTC TTT CAC CCA GTT | (SEQ ID NO:302) |
| TTT TTC CAT CCG GTT | (SEQ ID NO:258) | | TTC TTT CAC CCA GTC | (SEQ ID NO:303) |
| TTT TTC CAT CCG GTC | (SEQ ID NO:259) | | TTC TTT CAC CCA GTA | (SEQ ID NO:304) |
| TTT TTC CAT CCG GTA | (SEQ ID NO:260) | | TTC TTT CAC CCA GTG | (SEQ ID NO:305) |
| TTT TTC CAT CCG GTG | (SEQ ID NO:261) | 55 | TTC TTT CAC CCG GTT | (SEQ ID NO:306) |
| TTT TTC CAC CCT GTT | (SEQ ID NO:262) | | TTC TTT CAC CCG GTC | (SEQ ID NO:307) |
| TTT TTC CAC CCT GTC | (SEQ ID NO:263) | | TTC TTT CAC CCG GTA | (SEQ ID NO:308) |
| TTT TTC CAC CCT GTA | (SEQ ID NO:264) | | TTC TTT CAC CCG GTG | (SEQ ID NO:309) |
| TTT TTC CAC CCT GTG | (SEQ ID NO:265) | | TTC TTC CAT CCT GTT | (SEQ ID NO:310) |
| TTT TTC CAC CCC GTT | (SEQ ID NO:266) | 60 | TTC TTC CAT CCT GTC | (SEQ ID NO:311) |
| TTT TTC CAC CCC GTC | (SEQ ID NO:267) | | TTC TTC CAT CCT GTA | (SEQ ID NO:312) |
| TTT TTC CAC CCC GTA | (SEQ ID NO:268) | | TTC TTC CAT CCT GTG | (SEQ ID NO:313) |
| TTT TTC CAC CCC GTG | (SEQ ID NO:269) | | TTC TTC CAT CCC GTT | (SEQ ID NO:314) |
| TTT TTC CAC CCA GTT | (SEQ ID NO:270) | | TTC TTC CAT CCC GTC | (SEQ ID NO:315) |
| TTT TTC CAC CCA GTC | (SEQ ID NO:271) | 65 | TTC TTC CAT CCC GTA | (SEQ ID NO:316) |
| TTT TTC CAC CCA GTA | (SEQ ID NO:272) | | TTC TTC CAT CCC GTG | (SEQ ID NO:317) |
| TTT TTC CAC CCA GTG | (SEQ ID NO:273) | | TTC TTC CAT CCA GTT | (SEQ ID NO:318) |
| TTT TTC CAC CCG GTT | (SEQ ID NO:274) | | TTC TTC CAT CCA GTC | (SEQ ID NO:319) |
| TTT TTC CAC CCG GTC | (SEQ ID NO:275) | | TTC TTC CAT CCA GTA | (SEQ ID NO:320) |
| TTT TTC CAC CCG GTA | (SEQ ID NO:276) | 70 | TTC TTC CAT CCA GTG | (SEQ ID NO:321) |
| TTT TTC CAC CCG GTG | (SEQ ID NO:277) | | TTC TTC CAT CCG GTT | (SEQ ID NO:322) |
| TTC TTT CAT CCT GTT | (SEQ ID NO:278) | | TTC TTC CAT CCG GTC | (SEQ ID NO:323) |
| TTC TTT CAT CCT GTC | (SEQ ID NO:279) | | TTC TTC CAT CCG GTA | (SEQ ID NO:324) |
| TTC TTT CAT CCT GTA | (SEQ ID NO:280) | | TTC TTC CAT CCG GTG | (SEQ ID NO:325) |
| TTC TTT CAT CCT GTG | (SEQ ID NO:281) | 75 | TTC TTC CAC CCT GTT | (SEQ ID NO:326) |
| TTC TTT CAT CCC GTT | (SEQ ID NO:282) | | TTC TTC CAC CCT GTC | (SEQ ID NO:327) |
| TTC TTT CAT CCC GTC | (SEQ ID NO:283) | | TTC TTC CAC CCT GTA | (SEQ ID NO:328) |
| TTC TTT CAT CCC GTA | (SEQ ID NO:284) | | TTC TTC CAC CCT GTG | (SEQ ID NO:329) |
| TTC TTT CAT CCC GTG | (SEQ ID NO:285) | | TTC TTC CAC CCC GTT | (SEQ ID NO:330) |
| TTC TTT CAT CCA GTT | (SEQ ID NO:286) | 80 | TTC TTC CAC CCC GTC | (SEQ ID NO:331) |
| TTC TTT CAT CCA GTC | (SEQ ID NO:287) | | TTC TTC CAC CCC GTA | (SEQ ID NO:332) |
| TTC TTT CAT CCA GTA | (SEQ ID NO:288) | | TTC TTC CAC CCC GTG | (SEQ ID NO:333) |
| TTC TTT CAT CCA GTG | (SEQ ID NO:289) | | TTC TTC CAC CCA GTT | (SEQ ID NO:334) |
| TTC TTT CAT CCG GTT | (SEQ ID NO:290) | | TTC TTC CAC CCA GTC | (SEQ ID NO:335) |
| TTC TTT CAT CCG GTC | (SEQ ID NO:291) | 85 | TTC TTC CAC CCA GTA | (SEQ ID NO:336) |
| TTC TTT CAT CCG GTA | (SEQ ID NO:292) | | TTC TTC CAC CCA GTG | (SEQ ID NO:337) |
| TTC TTT CAT CCG GTG | (SEQ ID NO:293) | | TTC TTC CAC CCG GTT | (SEQ ID NO:338) |
| TTC TTT CAC CCT GTT | (SEQ ID NO:294) | | TTC TTC CAC CCG GTC | (SEQ ID NO:339) |
| TTC TTT CAC CCT GTC | (SEQ ID NO:295) | | TTC TTC CAC CCG GTA | (SEQ ID NO:340) |
| TTC TTT CAC CCT GTA | (SEQ ID NO:296) | 90 | TTC TTC CAC CCG GTG | (SEQ ID NO:341) |

Similarly, DNA sequences encoding peptides with all the shuffled sequences of SEQ ID NO:-1-SEQ ID NO:5 (that is, encoding the peptides SEQ ID NO:342-SEQ I NO:557 inclusive are included in the present invention, even though not written out individually.

DNA constructs encoding the present peptides and DNA constructs comprising one or more of SEQ ID NO:6-SEQ ID NO:341, inclusive, are preferably in a form suitable for replication in prokaryotic or eukaryotic unicellular host organisms such as bacteria or yeast, but also may be designed for introduction into the genome of eukaryotic cells (or cell lines) including mammalian cells. DNA constructs prepared for introduction into bacteria or yeast will include a replication system recognized by the host, the DNA sequence encoding the desired peptide, transcriptional and translational initiation regulatory sequences joined to the 5′-end of the DNA coding sequence and transcriptional and translational termination regulatory sequences joined to the 3′-end of the coding sequence. The transcriptional regulatory sequences may be employed which will include the replication system and transcriptional and translational regulatory sequences, together with an insertion site for the encoding DNA sequence.

Many such methods for recombinant production of the desired peptide or protein sequence are well known to the practitioner and may be applied to the production of the peptides of the invention without the exercise of inventive skill. See, for example, basic texts disclosing general methods of molecular biology, all of which are incorporated by reference, including: Sambrook, J. et al, Molecular Cloning: A Laboratory Manual, 2^{nd} Edition, Cold Spring Harbor Press, Cold Spring Harbor, N.Y., 1989; Ausubel, F. M. et al. Current Protocols in Molecular Biology, Vol. 2, Wiley-Interscience, New York, (current edition); Kriegler, Gene Transfer and Expression: A Laboratory Manual (1990); Glover, D. M., ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J. D. et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

The peptides may be purified, if necessary, using standard methods for physical, chemical or affinity separation which are well known in the art.

As noted above (for capping) and as described below, peptides of the present invention may include unconventional amino acids (e.g., norleucine). Moreover, modifications may provide a means for covalent attachment to a carrier or linker molecule.

Amino Acid Substitution and Addition Variants

Also included in this invention are peptides in which at least one amino acid residue and preferably, only one, has been removed and a different residue inserted in its place compared to the native sequence. For a detailed description of protein chemistry and structure, see Schulz, G. E. et al., Principles of Protein Structure, Springer-Verlag, New York, 1979, and Creighton, T. E., Proteins: Structure and Molecular Principles, W. H. Freeman & Co., San Francisco, 1984, which are hereby incorporated by reference. The types of substitutions which may be made in the peptide molecule of the present invention are conservative substitutions and are defined herein as exchanges within one of the following groups:

1. Small aliphatic, nonpolar or slightly polar residues: e.g., Ala, Ser, Thr, Gly;

2. Polar, negatively charged residues and their amides: e.g., Asp, Asn, Glu, Gln;

3. Polar, positively charged residues: e.g., His, Arg, Lys;

Pro, because of its unusual geometry, tightly constrains the chain. Substantial changes in functional properties are made by selecting substitutions that are less conservative, such as between, rather than within, the above groups (or two other amino acid groups not shown above), which will differ more significantly in their effect on maintaining (a) the structure of the peptide backbone in the area of the substitution (b) the charge or hydrophobicity of the molecule at the target site, or (c) the bulk of the side chain. Most substitutions according to the present invention are those that do not produce radical changes in the characteristics of the peptide molecule. Even when it is difficult to predict the exact effect of a substitution in advance of doing so, one skilled in the art will appreciate that the effect can be evaluated by routine screening assays, preferably the biological assays described below. Modifications of peptide properties including redox or thermal stability, hydrophobicity, susceptibility to proteolytic degradation or the tendency to aggregate with carriers or into multimers are assayed by methods well known to the ordinarily skilled artisan.

Chemical Derivatives of the Growth Inhibitory Peptides

“Chemical derivatives” of the peptides of this invention contain additional chemical moieties not normally a part of the peptide or polypeptide. Covalent modifications of the peptides are included within the scope of this invention. Such derivatized moieties may improve the solubility, absorption, biological half-life, and the like. Moieties capable of mediating such effects are disclosed, for example, in Remington's Pharmaceutical Sciences, 16^{th} ed., Mack Publishing Co., Easton, Pa. (1980) (or current edition).

Such modifications may be introduced into the molecule by reacting targeted amino acid residues with an organic derivatizing agent that is capable of reacting with selected side chains or terminal residues. Another modification is cyclization of the peptide or polypeptide.

Cysteinyl residues most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate (pH 5.5-7.0) which agent is relatively specific for the histidyl side chain. p-bromophenacyl bromide also is useful; the reaction is preferably performed in 0.1 M sodium cacodylate at pH 6.0.

Lysinyl and amino terminal residues are derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKₐ of the guanidine functional group. Furthermore, these reagents may react with the groups of lysine as well as the arginine ε-amino group.

Modification of tyrosyl residues permits introduction of spectral labels into a peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively.

Carboxyl side groups, aspartyl or glutamyl, may be selectively modified by reaction with carbodiimides (R—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues can be converted to asparaginyl and glutaminyl residues by reaction with ammonia.

Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention.

Derivatization with bifunctional agents is useful for cross-linking the peptide to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane.

Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for polypeptide or peptide immobilization.

Other modifications include hydroxylation of proline and lysine, phosphorylation of the hydroxyl groups of seryl or threonyl residues, methylation of the α-amino groups of lysine, arginine, and histidine side chains (T. E. Creighton, Proteins: Structure and Molecule Properties, W. H. Freeman & Co., San Francisco, pp. 79-86 (1983)), acetylation of the N-terminal amine, and, in some instances, amidation of the C-terminal carboxyl groups.

Also included are peptides wherein one or more D-amino acids are substituted for one or more L-amino acids.

Multimeric Peptides

The present invention also includes longer peptides built from repeating units of one or more of the peptides having the sequence KKKK (SEQ ID NO: 1), DDEEK (SEQ IS NO: 2), KLMSY (SEQ ID NO: 3), FFFKK (SEQ ID NO: 4) or FFHPV (SEQ ID NO: 5).

Such multimers (also termed “concatemers”) may be built from any of the peptides or their variants described herein. Moreover, a peptide multimer may comprise different combinations of the peptide monomers or addition variants thereof. Such oligomeric or multimeric peptides can be made by chemical synthesis or by recombinant DNA techniques as discussed herein. When produced by chemical synthesis, the oligomers preferably have from 2-12 repeats, more preferably 2-8 repeats of the core peptide sequence, and the total number of amino acids in the multimer preferably does not exceed about 110 residues (or their equivalents, when including linkers or spacers). Linkers can include enzymatically cleavable linkers that are know in the art. These may be engineered into a recombinant nucleic acid construct that encodes the multimer.

A preferred synthetic chemical peptide multimer has the formula

P¹ₙ

wherein P¹ is any one of KKKK (SEQ ID NO: 1), DDEEK (SEQ IS NO: 2), KLMSY (SEQ ID NO: 3), FFFKK (SEQ ID NO: 4) or FFHPV (SEQ ID NO: 5), shuffled sequence variants thereof (having the same amino acid composition in any and all permuted sequences) or biologically active substitution or addition variants of these peptides, wherein n=2-8, and wherein the peptide alone or in multimeric form has the biological activity of inhibiting cell proliferation, more particularly, cell proliferation mediated by abnormal activation or activity of PDGF-R, such as the autocrine activation present in NIH3T3-PDGF-ββ cells measured in an standard in vitro or in vivo bioassay of cell growth or proliferation.

In another embodiment, a preferred synthetic chemical peptide multimer has the formula

(P¹-Xₘ)ₙ-P²

P¹ and P² are peptides KKKK (SEQ ID NO: 1), DDEEK (SEQ IS NO: 2), KLMSY (SEQ ID NO: 3), FFFKK (SEQ ID NO: 4) or FFHPV (SEQ ID NO: 5) or addition variants of these pentapeptides,

wherein (a) P¹ and P² may be the same or different; moreover, each occurrence of P¹ in the multimer may be different a different one of the above five peptides (or variants);

(b) X is C₁-C₅ alkyl, C₁-C₅ alkenyl, C₁-C₅ alkynyl, C₁-C₅ polyether containing up to 4 oxygen atoms, wherein m=0 or 1 and n=1-7; X may also be Gly_{z} wherein, z=1-6,

and wherein the peptide alone or in multimeric form has the biological activity of inhibiting cell growth as described above.

When produced recombinantly, spacers are preferably Gly_{z} as described above, where z=1-6, and the multimers may have as many repeats of the core peptide sequence as the expression system permits, for example from two to about 100 repeats. A preferred recombinantly produced peptide multimer has the formula:

(P¹-Gly_{z})ₙ-P²

wherein:

(a) P¹ and P² are peptides KKKK (SEQ ID NO: 1), DDEEK (SEQ IS NO: 2), KLMSY (SEQ ID NO: 3), FFFKK (SEQ ID NO: 4) or FFHPV (SEQ ID NO: 5) or addition variants of these peptides, wherein P¹ and P² may be the same or different; moreover, each occurrence of P¹ in the multimer may be different peptides (or variant);

wherein n=1-100 and z=0-6;

and wherein the peptide alone or in multimeric form has the biological activity of inhibiting cell growth as described above.

The multimer is optionally capped at its N- and C-termini,

It is understood that such multimers may be built from any of the peptides or variants described herein. Although it is preferred that the addition variant monomeric units of the multimer have the biological activity described above, that is not necessary as long as the multimer to which they contribute has the activity.

As described above, peptides or peptide multimers of the present invention with potent growth inhibitory action allow the development of articles such as engineered biomedical implants for localized therapy of tumors following conventional resection protocols or for any type of implant when it is desirable to avoid attachment and growth of fibroblasts and smooth muscle cells that leads to fibrosis. A preferred example of such a device is a stent.

In one embodiment, the peptide or multimer is associated with, preferably chemically bonded by covalent or noncovalent linkages, to a solid (or carrier) surface including a synthetic polymer, natural polymer, or a combination thereof. Suitable synthetic polymers for the surface of an implant or other biomedical device include, but are not limited to, the following: poly(hydroxyethyl methacrylate), poly(ethylene terephthalate), poly(tetrafluoroethylene), fluorinated ethylene, poly(dimethyl siloxane), and combinations thereof.

Natural polymers suitable for fabricating a biomedical device may include, but are not limited to, the following: collagen, fibronectin, elastin, cellulose acetate, cellulose nitrate, polysaccharides, fibrin, gelatin, and combinations thereof.

Peptides, polypeptides or peptide multimers of the present invention may be attached or linked to a solid phase or matrix, preferably a polymer surface, by covalent bonding. Alternatively, the peptide, polypeptide or multimers may be bound noncovalently by Coulombic (electrostatic) or van der Waal forces or any combination thereof. Binding to a polymer surface, such as that of a biomedical device, may be direct or through a linker or spacer molecule. Alternatively, the peptide, polypeptide or multimer may be impregnated in or coated on the surface of a device. Coating may be accomplished, for example, by dipping, spraying or painting.

With respect to impregnation, the growth-inhibitory peptide can be incorporated into the polymeric material of a biomedical device during the process of synthesizing the polymer or fabricating the material. See, for example, Kang E T, et al., Macromolecules 296872-6879 (1996). In one example, the surface of an e biomedical device is formed of expanded polytetrafluoroethylene (ePTFE), one can mix into the extrudate used to make a polymeric layer of ePTFE a crystalline, particulate material like salt or sugar that is not soluble in a solvent used to form the extrudate. The extrudate solution is cast with particulate material into a film or sheet; and a second solvent, such as water, is applied to dissolve and remove the particulate material, thereby leaving a porous sheet. The porous sheet may then be placed into a solution containing one or more inhibitory peptides or multimers in order to fill the pores. Preferably, a vacuum is pulled on the film or sheet to insure that the applied peptide is received into the pores.

In another embodiment, the peptide may be present in a controlled release composition. In one example, the peptide may be encapsulated in a polymer. The polymeric matrix containing one or more peptides according to the present invention may include, without limitation, microparticles, microfibers or microfibrils. A microsphere could be contained within the mesh of fibrils connecting the matrix of nodes in ePTFE. Microparticles containing the peptide may be incorporated within or bound to a polymeric surface by adhesively positioning them onto the polymeric material. Alternatively, microparticles may be mixed with a fluid or gel and allowed to flow into the polymeric matrix of the surface. For peptide delivery, microfibers or microfibrils that have been loaded with peptide by extrusion can be adhesively layered or woven into the polymeric material included in a surface of a biomedical device.

In one embodiment, a peptide is bonded or linked to a carrier. A carrier, for purposes of this invention can be any of a number of materials, including synthetic or natural polymers, protein components of the extracellular matrix, polysaccharides, lipoproteins, immunoglobulins, or any combination thereof. The chemical coupling between the peptide and one of these macromolecules is generally achieved directly by reactive groups on the carrier substrate, the peptide, or the optional linker molecule. Reactive groups may either be a natural part of the carrier or the peptide or may be introduced by activating a reactive group in either molecule. Common reactive groups or functionalities include amino, imino, hydroxyl, sulfhydryl and carboxyl groups.

It may be advantageous to conjugate more than one type of peptide or peptide multimer to a particular carrier, such as a synthetic polymeric surface of a biomedical device.

In one embodiment of the present medical device, the natural and/or synthetic polymer(s) forming the device are biostable or bioabsorbable. When the device is biostable, the peptide may diffuse out from the biostable material in which it is incorporated. If, however, the polymer is bioabsorbable, the incorporated peptide may be delivered to an intended site in part by the process of degradation and resorption of the polymer itself.

While biological polymers such as fibrin, collagen and elastin possess high biocompatibility per se, their mechanical properties are often inadequate and their cost of production is generally much higher than synthetic polymers. Therefore, synthetic and biological polymers may be combined to produce a biomedical device having superior mechanical properties that are a result of a synthetic component and the biocompatibility that is the result of the biological component. Blending techniques are well known. See, for example, International Journal of Artificial Organs 14:295-303 (1991).

Cell Adhesion Resisting (CAR) Surfaces

A “cell-adhesion resisting” or “cell-adhesion resistive” (“CAR”) material or agent, when coated onto a solid surface, inhibits or prevents cell adherence or attachment to the surface. Based on the properties of these materials, certain macromolecules are also less likely to bind to a CAR surface. According to the present invention, a growth-inhibitory peptide may be provided in the form of a surface of an article or device; cell growth would be inhibited by the properties which have been conferred on the surface. Suitable CAR materials include but are not limited to polyethylene glycol, glyme and derivatives thereof, poly-HEMA, poly-isopropylacrylamide and, preferably any of a number of polysaccharides including hyaluronic acid (HA) and alginic acid (AA). In a more preferred embodiment, HA is used as a CAR material. In general, highly hydrophilic substances containing a high concentration of hydroxyl groups may be used as CAR materials, either alone or in combination.

A CAR region is an area on a surface onto which a CAR material has been placed, added, spotted, dropped, etc. A first region is “juxtaposed” to a second region if the two regions are adjacent to one another on a surface, or, are sufficiently close to one another that cells in or on the first region can respond to signals the second, juxtaposed region. Two juxtaposed regions may be in direct contact so that no other surface intervenes, or may be spaced at varying distances from one another. (See, commonly assigned U.S. patent application Ser. No. ______, John J. Hemperly, “Proliferation and Differentiation of Stem Cells Using Extracellular Matrix and Other Molecules,” filed on even date herewith and based on U.S. Provisional application No. 60/326,440, all of which are incorporated by reference in their entirety.)

Methods and compositions useful for creating CAR layers and CAR surfaces are described in greater detail in copending commonly assigned U.S. patent application Ser. No. ______, Liebmann-Vinson et al., “Cell Adhesion Resisting Surfaces” filed on even date herewith and hereby incorporated by reference in its entirety, as well as in references cited therein.

Therapuetic Compositions and Uses

As noted above, the present invention embodies a method of treating a subject suffering from a cell proliferative disorder, including, but not limited to cancer. The method is well-suited to treat a condition in which the cells affected by the disorder have abnormal or inappropriate PDGF-R activity. In one embodiment, cells of a subject characterized as having inappropriate PDGF-R activity are contacted with a peptide or multimer of this invention or with a nucleic acid molecule encoding such a peptide or multimer, as a way to inhibit their growth and thereby treat the associated disease or condition.

As noted, the peptide or multimer used in the treatment method may be chemically bonded, bound, or linked to, or otherwise associated with, a biomedical implant that comprises a natural or synthetic polymer (or combination of both) as described above. The treatment method may further comprise administering to the subject a therapeutically effective amount of a conventional agent known to be useful for treating the subject's disease or disorder. Thus, in the case of cancer, this additional agent may be a known anti-cancer drug or biologic agent. For example, a subject in need of such treatment is administered or subjected to a therapeutic composition or biomedical device that comprises the present growth-inhibitory peptide or peptide multimer in an amount effective to inhibit PDGF-R activity, the composition or device being administered in combination with a cytotoxic agent, e.g., VP-16 or cisplatin. Other suitable agents for use in combination with the present peptides include: cyclophosphamide, enoxaprin, angiopeptin, endostatin, paclitaxel, 5-fluorouracil, vinblastine, vincristine, an epothilone, angiostatin, hirudin, acetylsalicylic acid, a thymidine kinase inhibitor, or a combination thereof.

The preferred animal subject of the present invention is a mammal. By the term “mammal” is meant an individual belonging to the class Mammalia. The invention is particularly useful in the treatment of human subjects.

By the term “treating” is intended the administering to subjects the compositions of this invention for purposes which may include prevention, amelioration, or cure of a disease or disorder.

The therapeutic or pharmaceutical composition of the present invention may be comprised of the polypeptide, peptide, combination or multimer and a pharmaceutically

Administration may be by parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

Compositions within the scope of this invention include all compositions wherein the peptide, polypeptide or multimer contained in an amount effective to achieve its intended purpose. While individual needs vary, determination of optimal ranges of effective amounts of each component is within the skill of the art. Typical dosages comprise 1 ng/kg body weight to 100 mg/kg/body wt. The preferred dosages comprise 1 μg/kg body weight to 10 mg/kg/body wt.

In addition to the pharmacologically active compounds, the pharmaceutical compositions preparations may contain suitable pharmaceutically acceptable carriers comprising excipients and auxiliaries which facilitate processing of the active compounds into preparations which can be used pharmaceutically. Preferably, the preparations, particularly those preparations which can be administered orally and which can be used for the preferred type of administration, such as tablets, dragees, and capsules as well as suitable solutions for administration by injection or orally, contain from about 0.01 to 99 percent, preferably from about 20 to 75 percent of active compound(s), together with the excipient.

The pharmaceutical formulation for systemic administration according to the invention may be formulated for enteral, parenteral or topical administration. Indeed, all three types of formulation may be used simultaneously to achieve systemic administration of the active ingredient.

Suitable formulations for oral administration include hard or soft gelatin capsules, dragees, pills tablets, including coated tablets, elixirs, suspensions, syrups or inhalations and controlled release forms thereof. Solid dosage forms in addition to those formulated for oral administration include rectal suppositories. The composition may also be administered in the form of an implant, as noted herein.

Suitable formulations for topical administration include creams, gels, jellies, mucilages, pastes and ointments. The compounds may also be formulated for transdermal administration, for example, in the form of transdermal patches so as to achieve systemic administration.

Suitable injectable solutions include intravenous subcutaneous and intramuscular injectable solutions. The compound may also be administered in the form of an infusion solution or as a nasal inhalation or spray.

Suitable excipients are well-known in the art. See for example Remington's Pharmaceutical Sciences, 16^{th} ed., Mack Publishing Co., Easton, Pa. (1980) or more recent updated editions.

As described above, unwanted cell proliferation may result from inappropriate PDGF-R activity occurring in different types of cells such as cancer cells, stromal cells surrounding a cancer cell, endothelial cells, and smooth muscle cells. Thus the present method for treating a subject with a solid tumor characterized by inappropriate PDGF receptor activity may include contacting not only cancer cells but also cells stromal cells and other neighboring cells with the growth inhibitory peptides or peptide multimers.

In one embodiment, the treatment method includes surgical removing of some or all of a solid tumor followed by treatment with the peptide, preferably by implanting the biomedical device of the invention proximal to the surgical site. The device has associated with it the growth inhibitory peptide or multimer that is made available for interaction with cells at or near the surgical site by virtue of the peptide or multimer's release from the device or their action while linked or associated with the device.

Having now generally described the invention, the same will be more readily understood through reference to the following examples that are provided by way of illustration, and are not intended to be limiting of the present invention, unless specified.

### EXAMPLE 1

### Cell Line

NIH 3T3 cells transfected with PDGF-ββ (a stable cell line) were obtained from Mount Sinai School of Medicine. These cells overexpress PDGF-ββ and are activated via the PDGF-R in an autocrine fashion. Cells were grown at 37° C. in Dulbecco's Modified Eagles Medium (high glucose) (DMEM) with 10% heat-inactivated fetal calf serum (FCS) with 100 units or μg/ml penicillin/streptomycin with 750 μg/ml G418 sulfate (Geneticin) selection. Cells were incubated in an atmosphere of 5% CO₂, and cultures were fed twice weekly. For subcultivation, cells were allowed to attain confluence and washed twice with PBS or Hank's balanced salt solution (minus Ca⁺⁺ and Mg⁺⁺) before addition of a trypsin/EDTA solution to dislodge the cells. Cells were split anywhere from ¼ to 1/12 into a sterile T-75 Flask depending on the time desired until confluence.

### EXAMPLE 2

### Peptide Screening

To identify peptides that inhibit cell growth in culture, candidate peptides were screened in a growth assay with NIH3T3-PDGF-ββ cells. Cells were expanded in DMEM containing G418 as described above. Following trypsinization, cells were counted and plated into 96 well plates at the desired density (generally 6×10³ cells/well in 250 μl medium in DMEM supplemented with 10% FCS (G418 was omitted as it can interfere with subsequent assays). Peptides were added when the cells reached approximately 50-75% of confluency. Peptides (purchased from Bachem or Sigma) were reconstituted with water and lyophilized prior to use. Peptides were prepared in BITS medium (DMEM supplemented with 0.5% BSA, 1× Insulin/Transferrin/Selenium (1× ITS) which resulted in final concentrations of 0.01 g/L insulin, 0.007 mg/L sodium selenite, 0.006 g/L transferrin and 0.002 g/L ethanolamine) at peptide concentrations ranging from 1-12 mM as indicated in Tables 1 and 2. Growth medium was removed and the peptide solution added (250 μl/well). Cells were incubated for 5 days without feeding prior to testing. After this time, growth of cells treated with each peptide was compared to growth in control medium (no peptide added).

Cell number was assessed by measurement of total cellular double-stranded DNA using the PicoGreen Assay Kit (Molecular Probes, Eugene, Oreg., USA, lot #6405-1). For PicoGreen analysis, cell lysates (100 μl ) were added to 100 82 l of the dye solution prepared by diluting the PicoGreen dye (1:200 in 1× TE according to the manufacturer's instructions). Plates were read after five minutes in a fluorimeter (irradiated at an excitation wavelength of 485 nm). Fluorescence emission was measured at 530 nm using a CytoFluor Series 4000 (PerSeptive Biosystems, Framingham, Mass.). For correlating DNA level to cell number, a standard curve was established for the NIH3T3-PDGF-ββ cells. For analysis, DNA absorbance/emission was compared to the absorbance/emission shown by a standard curve of DNA.

Using this method, several peptides were identified, in the initial screen of the library, as inhibiting growth of the NIH3T3-PDGF-ββ cells. These peptides included KKKK (SEQ ID NO: 1), DDEEK (SEQ ID NO: 2), KLMSY (SEQ ID NO: 3), FFFKK (SEQ ID NO: 4), and FFHPV (SEQ ID NO: 5). The results of these experiments are described in the Examples 3 and 4 below (see Tables 1 and 2).

TABLE 1
Effect of varying concentrations of KKKK (SEQ ID NO: 1)
on NIH3T3 Cells Stimulated by PDGF ββ

| Group | | % of control cell growth |
|---|---|---|
| DMEM-10% FCS | | 100 |
| DMEM-BITS | | 100 |
| KKKK | 12 mM | 39 |
| KKKK | 6 mM | 65 |
| KKKK | 3 mM | 78 |

TABLE 2
Effect of Inhibitory Peptides on NIH3T3 Cells Stimulated by PDGF ββ

| | SEQ ID | Conc | % of control |
|---|---|---|---|
| Peptide | NO: | (mM) | cell growth |
| Control (DMEM − 10% FCS | | | 100 |
| Control (DMEM + BITS) | | | 100 |
| DDEEK | 2 | 12 | 16 |
| | | 6 | 11 |
| | | 3 | 93 |
| | | 1 | 108 |
| KLMSY | 3 | 12 | 10 |
| | | 6 | 76 |
| | | 3 | 126 |
| | | 1 | 127 |
| FFFKK | 4 | 12 | 18 |
| | | 6 | 69 |
| | | 3 | 103 |
| | | 1 | 111 |
| FFHPV | 5 | 9.5 | 16 |
| | | 6 | 85 |
| | | 3 | 111 |
| | | 1 | 116 |
| KKKK | 1 | 12 | 17 |
| | | 6 | 41 |
| | | 3 | 72 |
| | | 1 | 105 |

### EXAMPLE 3

### Activity of KKKK (SEQ ID NO:1)

The results in this example demonstrate that a peptide with the sequence KKKK (Bachem) was an effective inhibitor of the growth of NIH3T3-PDGF-ββ cells. For analysis of DNA, absorbance/emission of control and experimental wells was compared to the absorbance/emission shown by a DNA standard curve to calculate cell members which were converted to % of control cell growth as presented in Table. These data indicate that a 12 mM concentration of KKKK inhibited cell growth by approximately 61% compared to control medium (BITS control). Peptide at 6 mM produced a 36% inhibition whereas 3 mM peptide gave a 21% inhibition. It is noted that the base medium, (BITS) into which the peptide was added for screening, is a medium which does not contain hydrolysate. In contrast, the 10% control value represents DMEM medium used for expanding the cells (which is a hydrolysate-based medium that included 10% FCS.

### EXAMPLE 4

### Activity of Inhibitory Pentapeptides

Results shown in Table 2, % of control cell growth, were obtained using a PicoGreen assay. The peptide KKKK (SEQ ID NO: 1) (from Sigma) inhibited cell growth to an extent greater than that observed in Example 3 (same peptide sequence, different source). Here, 6 mM peptide gave 59% inhibition when compared to the base medium (BITS). This compares with 36% inhibition in Example 3. Moreover, a 12 mM peptide gave 82% inhibition when compared to the base medium alone, whereas the same peptide gave 61% inhibition in Example 3.

A second peptide obtained from Bachem, FFHPV (SEQ ID NO: 5), produced 14% inhibition compared to base medium at a 6 mM and an 82% inhibition at 9.5 mM.

A third peptide, FFFKK (SEQ ID NO: 4) from Bachem exhibited 29% inhibition and 82% inhibition at 6 mM and 12 mM concentrations, respectively, as compared to the base medium alone.

The peptide KLMSY (SEQ ID NO: 3) from Bachem gave 23% and 91% inhibition at 6 mM and 12 mM, respectively, as compared to the base medium.

Finally, the peptide DDEEK (SEQ ID NO: 2) from Bachem exhibited 91% and 84% inhibition at 6 mM and 12 mM concentrations, respectively, as compared to the base medium alone.

### EXAMPLE 4

### Predicted Parametric Space that Includes Range of Pentapeptides with Predicted Inhibitory Activity

The following are the parameters for the five peptides described herein.

| Peptide | SEQ ID NO: | Total charge | MlogP | MW | Dipole |
|---|---|---|---|---|---|
| DDEEK | 2 | −3 | −6.69 | 631 | 38.3 |
| KLMSY | 3 | 1 | −1.2 | 641 | 129.2 |
| FFFKK | 4 | 2 | 0.63 | 717 | 40 |
| FFHPV | 5 | 0 | 0.23 | 645 | 53.4 |
| KKKK | 1 | +4 | −1.85 | 534 | 81 |

Pentapeptides most similar in properties to DDEEK would be preferred as this appears to be the most potent in inhibiting cell growth. A preferred range of charge would be +2 to −3. A preferred MW ranging would be from 631-717 Da. A preferred range in MlogP is between about −8.5 and −2, more preferably between about −7 and −3.5. Preferred dipole range is 38-129

Closer examination shows a correlation between inhibitory activity and property space.

At 6 mM concentrations, DDEEK was the most potent inhibitor. Interestingly this peptide is in a distinct property space in a 2 dimensional space defined by hydrophilicity (lipophilicity) and charge. Thus, all peptides in this space, that have charges of between −2 and −4, and lipophilicity of between about −2 and −8 constitute a group that are predicted to have potent inhibitory activity and are within the scope of this invention. Some of these peptides fit in the group defined as shuffled sequences of DDEEK (SEQ ID NO:2), i.e., peptides with amino acid sequences SEQ ID NO: 342-SEQ ID NO:370 as set forth above. This is also shown in FIG. 1.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. An isolated peptide or polypeptide of no more than about 50 amino acid residues which, when contacted with cells in which a PDGF-R is activated in an autocrine manner, inhibits the growth of said cells, wherein said peptide or polypeptide comprises one or more amino acid sequences selected from the group consisting of KKKK (SEQ ID NO: 1), DDEEK (SEQ ID NO: 2), KLMSY (SEQ ID NO: 3), FFFKK (SEQ ID NO: 4), FFHPV (SEQ ID NO: 5), or (i) a combination thereof, (ii) a biologically active variant thereof having the same amino acid composition in a different sequence, (iii) or a biologically active substitution or addition variant.

2. The peptide or polypeptide of claim 1 having no more than about 20 amino acids.

3. The peptide of claim 2 having no more than about 10 amino acids.

4. The peptide of claim 1 which is selected from the group of peptides consisting of KKKK (SEQ ID NO: 1), DDEEK (SEQ ID NO: 2), KLMSY (SEQ ID NO: 3), FFFKK (SEQ ID NO: 4), and FFHPV (SEQ ID NO: 5).

5. The peptide of claim 4 having five amino acids and the sequence DDEEK (SEQ ID NO: 2).

6. A pentapeptide that falls within a parameter space defined by at least two physicochemical parameters of peptides SEQ ID NO:2-SEQ ID NO:5, that has the following properties:
(a) inhibits the growth of cells that expressing a PDGF-R that is activated for growth in an autocrine manner;
(b) total charge of between −4 and +2; and
(c) MlogP value between about −8.5 and −2.

7. The pentapeptide of claim 6 having a total charge between −4 and −2, and a MlogP value between about −7 and −3.5.

8. A chemically synthesized peptide multimer comprising the peptide or variant of claim 1, which multimer is selected from the group consisting of:
(a) a multimer having the formula P¹ₙ wherein
(i) P¹ is any one of KKKK (SEQ ID NO: 1), DDEEK (SEQ IS NO: 2), KLMSY (SEQ ID NO: 3), FFFKK (SEQ ID NO: 4) or FFHPV (SEQ ID NO: 5), a shuffled sequence variant thereof having the same amino acid composition in any sequence, or a substitution or addition variant thereof, and
(ii) n=2-8,
(b) a multimer having the formula (P¹-Xₘ)ₙ-P², wherein
(i) P¹ and P² are peptides KKKK (SEQ ID NO: 1), DDEEK (SEQ IS NO: 2), KLMSY (SEQ ID NO: 3), FFFKK (SEQ ID NO: 4) or FFHPV (SEQ ID NO: 5), shuffled sequence variant thereof, or a substitution or an addition variant thereof,
(ii) P¹ and P² are the same or different peptides;
(iii) X iS C₁-C₅ alkyl, C₁-C₅ alkenyl, C₁-C₅ alkynyl, C₁-C₅ polyether containing up to 4 oxygen atoms;
(iv) m=0 or 1; and
(v) n=1-7,
wherein the peptide multimer has the biological activity of inhibiting cell proliferation mediated by autocrine activation of PDGF-R measured in an standard in vitro or in vivo bioassay of cell growth or proliferation.

9. A recombinantly produced peptide multimer comprising the peptide or variant of claim 2, which multimer has the formula (P¹-Gly_{z})ₙ-P², wherein:
(i) P¹ and P² are peptides KKKK (SEQ ID NO: 1), DDEEK (SEQ IS NO: 2), KLMSY (SEQ ID NO: 3), FFFKK (SEQ ID NO: 4) or FFHPV (SEQ ID NO: 5), a shuffled sequence variant thereof, or a substitution or an addition variant thereof,
(ii) P¹ and P² are the same or different;
(iii) z=0-6; and
(iv) n=1-100.
wherein the peptide multimer has the biological activity of inhibiting cell proliferation mediated by autocrine activation of PDGF-R measured in an standard in vitro or in vivo bioassay of cell growth or proliferation.

10. An isolated nucleic acid molecule encoding (a) the polypeptide or peptide of claim 1 or any permuted sequence of SEQ ID NO:2-SEQ ID NO:5, or (b) the peptide multimer of claim 9.

11. The nucleic acid molecule of claim 10 comprising one or more of SEQ ID NO:6-SEQ ID NO:341, inclusive.

12. An expression vector comprising the nucleic acid molecule of claim 10 operatively linked to:
(a) a promoter, and
(b) optionally, additional regulatory sequences that regulate expression of said nucleic acid in a eukaryotic cell,
which vector is capable of expressing said peptide in a host cell.

13. An expression vector comprising the nucleic acid molecule of claim 11 operatively linked to:
(a) a promoter, and
(b) optionally, additional regulatory sequences that regulate expression of said nucleic acid in a eukaryotic cell,
which vector is capable of expressing said peptide in a host cell.

14. The expression vector of claim 12 which is a plasmid.

15. The expression vector of claim 12 which is a viral vector.

16. A solid phase article comprising the polypeptide or peptide of claim 1 in contact with a solid surface.

17. A solid phase article comprising the multimer of claim 8 or 9 associated with or chemically linked to a solid surface.

18. The article of claim 16 wherein said solid surface comprises a synthetic polymer, natural polymer, or a combination thereof.

19. The article of claim 16 further comprising an additional layer of a cell adhesion resistant material between said polypeptide or peptide and said surface.

20. The article of claim 16 wherein said cell adhesion resistant material is material selected from the group consisting of (a) polyethylene glycol, (b) glyme, (c) a glyme derivative, (d) poly-HEMA, (e) polyisopropylacrylamide, (f) hyaluronic acid, (g) alginic acid and (h) a combination of any of (a)-(g).

21. The article of claim 16 wherein said solid surface comprises a synthetic polymer selected from the group consisting of poly(hydroxyethyl methacrylate), poly(ethylene terephthalate), poly(tetrafluoroethylene), fluorinated ethylene, poly(dimethyl siloxane), and a combination thereof.

22. The article of claim 16 wherein said solid surface comprises a natural polymer selected from the group consisting of collagen, fibronectin, elastin, cellulose acetate, cellulose nitrate, polysaccharides, fibrin, gelatin, and a combination thereof.

23. The article of claim 16 wherein said peptide is chemically linked to said surface through a linker molecule.

24. A biomedical device for inhibition of abnormal or undesired cell attachment, cell growth or both attachment and growth, comprising a biocompatible surface having chemically and/or physically associated with said surface a proliferation inhibiting amount of the peptide, polypeptide or combination of claims 1 or a nucleic acid molecule encoding said peptide or polypeptide.

25. A biomedical device for inhibition of abnormal or undesired cell attachment, cell growth or both attachment and growth, comprising a biocompatible surface having chemically and/or physically associated with said surface a proliferation inhibiting amount of a multimer of claim 8 or 9 or a nucleic acid molecule encoding said multimer.

26. The device of claim 24 further comprising an additional layer of a cell adhesion resistant material between said polypeptide or peptide and said surface.

27. The device of claim 24 wherein said peptide or polypeptide is impregnated in or coated on said surface.

28. The device of claim 24 wherein said peptide or polypeptide is present in a controlled release composition.

29. A therapeutic composition that inhibits the undesired growth of cells mediated by abnormal activation or activity of PDGF-R, comprising
(a) the peptide, polypeptide or combination of claim 1; and.
(b) a therapeutically acceptable carrier or excipient.

30. A therapeutic composition that inhibits the undesired growth of cells mediated by abnormal activation or activity of PDGF-R, comprising
(a) the multimer of claim 8 or 9; and.
(b) a therapeutically acceptable carrier or excipient.

31. The therapeutic composition of claim 29 wherein said abnormal activation comprises autocrine activation of the PDGF-R.

32. A therapeutic composition that inhibits the undesired growth of cells mediated by abnormal activation or activity of PDGF-R, comprising
(a) the expression vector of claim 12; and
(b) a therapeutically acceptable carrier or excipient.
wherein expression of said nucleic acid molecule results in production and growth inhibitory action of said peptide.

33. A method of inhibiting cell proliferation comprising contacting cells undergoing undesired proliferation with an effective amount of the peptide, polypeptide or combination of claim 1.

34. The method of claim 33 wherein the cell being inhibited is a tumor or cancer cell.

35. The method of claim 34 wherein the tumor or cancer cell is a carcinoma cell, an osteocarcinoma cell, a sarcoma cell, an osteosarcoma cell, a glioma cell, a melanoma cell, a myxoma cell, an adenoma cell, a neuroblastoma cell, or a rhabdomyoma-derived cell.

36. The method of claim 33 wherein the cell being inhibited is a lung cell, a breast cell, a colon cell, a prostate cell, a kidney cell, an ovary cell, a testicular cell, a skin cell, a pancreatic cell, a thyroid cell, an adrenal cell, a pituitary cell, a brain cell, a muscle cell or a bone cell.

37. The method of claim 33 wherein said contacting is in vivo in a subject.

38. The method of claim 33 wherein said contacting is in vitro.

39. The method of claim 33 which further comprises, administering to the subject a therapeutically effective amount of one or more agents or drugs selected from the group consisting of cisplatin, cyclophosphamide, VP-16, enoxaprin, angiopeptin, endostatin, paclitaxel, 5-fluorouracil, vinblastine, vincristine, an epothilone, angiostatin, hirudin, acetylsalicylic acid, and a thymidine kinase inhibitors.

40. A method of treating a subject suffering from a cell proliferative disorder, comprising contacting cells of said subject which are characterized by inappropriate PDGF receptor activity with an effective amount of a peptide, polypeptide or combination according to claim 1 or with a nucleic acid molecule encoding said peptide or polypeptide, which nucleic acid is expressible in said cells.

41. The method of claim 40 wherein said peptide or polypeptide is associated or chemically linked to a biomedical implant.

42. The method of claim 41 wherein said biomedical implant comprises at least one of a natural polymer or a synthetic polymer.

43. The method of claim 40, further comprising administering to the patient a therapeutically effective amount of a composition comprising an agent selected from the group consisting of cisplatin, cyclophosphamide, VP-16, enoxaprin, angiopeptin, endostatin, paclitaxel, 5-fluorouracil, vinblastine, vincristine, epothilones, angiostatin, hirudin, acetylsalicylic acid, thymidine kinase inhibitors, and combinations thereof.

44. A method of treating a subject who has a solid tumor, the cells of which are characterized by inappropriate PDGF receptor activity, the method comprising contacting tumor cells and/or cells surrounding tumor cells of said subject with an effective amount of a peptide, polypeptide or combination according to claim 1 or a with a nucleic acid molecule encoding said peptide or polypeptide which nucleic acid is expressible in said tumor or surrounding cells.

45. The method of claim 44 wherein said peptide or polypeptide is associated with or chemically linked to a biomedical implant.

46. The method of claim 45 wherein said biomedical implant comprises at least one of a natural or synthetic polymer.

47. The method of claim 44 further comprising administering to the subject a therapeutically effective amount of a composition comprising an agent selected from the group consisting of cisplatin, cyclophosphamide, VP-16, enoxaprin, angiopeptin, endostatin, paclitaxel, 5-fluorouracil, vinblastine, vincristine, epothilones, angiostatin, hirudin, acetylsalicylic acid, thymidine kinase inhibitors, and combinations thereof.

48. The method of claim 44, further comprising prior to said contacting step, the steps of
(i) surgically removing or debulking said solid tumor; and
(ii) implanting a biomedical device proximal to the site of the surgery,
said device having associated therewith and available for interaction with cells surrounding said site, a synthetic or recombinant peptide or polypeptide of no more than about 50 amino acid residues which, when contacted with cells in which a PDGF-R is activated in an autocrine manner, inhibits the growth of said,wherein said peptide or polypeptide comprises an amino acid sequence selected from the group consisting of KKKK (SEQ ID NO: 1), DDEEK (SEQ ID NO: 2), KLMSY (SEQ ID NO: 3), FFFKK (SEQ ID NO: 4), FFHPV (SEQ ID NO: 5), and combinations thereof, or with a nucleic acid molecule encoding said peptide or polypeptide.

49. The method of claim 48, wherein said peptide or polypeptide associated with said device has no more than about 20 amino acids.

50. The method of claim 49 wherein said peptide or polypeptide associated with said device has no more than about 10 amino acids.

51. The method of claim 50 wherein said peptide associated with said device is a pentapeptide selected from the group consisting of KKKK (SEQ ID NO: 1), DDEEK (SEQ ID NO: 2), KLMSY (SEQ ID NO: 3), FFFKK (SEQ ID NO: 4), and FFHPV (SEQ ID NO: 5).

52. The method of any of claims 48 further comprising administering to the subject a therapeutically effective amount of a composition comprising an agent selected from the group consisting of cisplatin, cyclophosphamide, VP-16, enoxaprin, angiopeptin, endostatin, paclitaxel, 5-fluorouracil, vinblastine, vincristine, epothilones, angiostatin, hirudin, acetylsalicylic acid, thymidine kinase inhibitors, and combinations thereof.

53. A method of inhibiting proliferation of a cell of mesenchymal origin in vivo, the method comprising administering to a subject in which said cells are present a proliferation-inhibiting effective amount of a peptide, polypeptide or combination of claim 1, or a nucleic acid molecule encoding said peptide or polypeptide.
